(19) United States
(12) Patent Application Publication
(10) Pub. No.: US 2010/0092556 A1
(43) Pub. Date: Apr. 15, 2010

(54) ALFUZOSIN FORMULATIONS, METHODS OF MAKING, AND METHODS OF USE

(76) Inventors: Kristin Arnold, Morrisville, PA (US); Gandha V. Naringrekar, Princeton, NJ (US); Siva Ram K. Nutalapati, Princeton, NJ (US); Zhongshui Yu, Bridgewater, NJ (US)

Correspondence Address: CANTOR COLBURN, LLP, 20 Church Street, 22nd Floor, Hartford, CT, 06103, US

(21) Appl. No.: 12/419,721

(22) Filed: Apr. 7, 2009

Related U.S. Application Data
(63) Continuation-in-part of application No. 11/953,181, filed on Dec. 10, 2007.
(60) Provisional application No. 61/042,931, filed on Apr. 7, 2008.
(60) Provisional application No. 61/144,608, filed on Jan. 14, 2009.
(60) Provisional application No. 60/869,464, filed on Dec. 11, 2006.
(60) Provisional application No. 60/944,920, filed on Jun. 19, 2007.

(51) Int. Cl. A61K 9/24; A61K 31/517; A61P 13/08; A61P 13/10

(52) U.S. Cl. 424/472; 514/266.24

(57) ABSTRACT

Alfuzosin compositions comprising a tablet core of alfuzosin and a release-retarding matrix comprising about 40 to about 80% (by weight) hydroxypropyl methyl cellulose with a maximum apparent viscosity of about 5600 cP, based on the total weight of the tablet core; and an extended-release coating substantially surrounding the tablet core comprising a release-retarding coating material, wherein the compositions are bioequivalent to the reference dosage form of NDA #021287 (UROXATRAL) are disclosed. Methods of making and using the alfuzosin compositions are also disclosed.

### CROSS-REFERENCE TO RELATED APPLICATIONS

This application claims the benefit of U.S. Provisional Application Nos. 61/042,931, filed Apr. 7, 2008, and 61/144,608, filed Jan. 14, 2009, and is a continuation-in-part of U.S. patent application Ser. No. 11/953,181, filed Dec. 10, 2007, which claims the benefit of U.S. Provisional Application Nos. 60/869,464, filed Dec. 11, 2006, and 60/944,920, filed Jun. 19, 2007; each of the above-mentioned applications is incorporated by reference herein in its entirety.

### BACKGROUND

This application relates to improved alfuzosin compositions for therapeutic purposes and methods of making and using the alfuzosin compositions.

Alfuzosin, (R,S)—N-[3-[(4-amino-6,7-dimethoxy-2-quinazolinyl)methylamino]propyl]tetrahydro-2-furancarboxamide, is a selective antagonist of post-synaptic alpha1-adrenoreceptors, which are located in the prostate, bladder base, bladder neck, prostatic capsule, and prostatic urethra.

Alfuzosin hydrochloride is a white to off-white crystalline powder that melts at approximately 240° C. It is freely soluble in water, sparingly soluble in alcohol, and practically insoluble in dichloromethane. U.S. Pat. No. 4,315,007 discloses a process for preparing alfuzosin or its HCl. Mono-, di-, tri-, and tetrahydrates of alfuzosin HCl are disclosed in U.S. Pat. No. 5,545,738.

Alfuzosin hydrochloride is marketed in the United States under the brand name UROXATRAL® and elsewhere as XATRAL. The dosage form currently approved for marketing in the United States is a 10 mg extended release oral tablet (approved for marketing by the US Food and Drug Administration (FDA) under New Drug Application (NDA) #021287 on Jun. 12, 2003). The UROXATRAL® 10 mg extended-release tablet is a round trilayer tablet comprising as inactive ingredients: colloidal silicon dioxide (NF), ethylcellulose (NF), hydrogenated castor oil (NF), hydroxypropyl methylcellulose (USP), magnesium stearate (NF), mannitol (USP), microcrystalline cellulose (NF), povidone (USP), and yellow ferric oxide (NF). The three-layer tablet has one white layer between two yellow layers and is debossed with X10.

UROXATRAL® tablets are approved for the treatment of the signs and symptoms of benign prostatic hyperplasia. Additional disclosed uses for alfuzosin are management of blood pressure (U.S. Pat. No. 6,096,339), delaying onset of male ejaculation (U.S. Pat. No. 5,922,341), treating premature ejaculation (U.S. Pat. No. 5,707,999), treating female sexual dysfunction (US 2004/132697); treating lower urinary tract symptoms (US 2004/072850), and treating primary dysmenorrhea (US 2006/128719).

The pharmacokinetics of UROXATRAL have been evaluated in adult healthy make subjects. Under fed conditions following multiple dosing of 10 mg UROXATRAL, Cmax is 13.6±5.6 (s.d.) ng/mL with a Tₘₐₓ of 8 hrs and AUC0-24 of 194±75 (s.d.) ng-hr/mL. These stead-state values are reported to be 1.2 to 1.6 fold higher than those observed after a single administration, corresponding to a Cmax of 8.5-11.3 ng/mL. The extent of absorption of UROXATRAL is also reported to be 50% lower under fasting conditions than under fed (non-fasted) conditions (UROXATRAL prescribing information, Sanofi-Aventis, U.S., LLC, August 2007).

The recommended dosage is one 10 mg alfuzosin HCl extended-release tablet daily, to be taken immediately after the same meal each day. The UROXATRAL® extended-release tablets should not be chewed or crushed.

Following oral administration, alfuzosin undergoes extensive metabolism by the liver, with only 11% of the administered dose excreted unchanged in the urine. Three metabolic pathways metabolize alfuzosin: oxidation, O-demethylation, and N-dealkylation. The metabolites are not pharmacologically active. CYP3A4 is the principal hepatic enzyme isoform involved in its metabolism.

U.S. Pat. No. 6,149,940 ('940 patent) discloses a preparation of an alfuzosin 10 mg once daily composition for oral delivery using a technology termed GEOMATRIX that has been developed by Jagotec-AG. The three-layer GEOMATRIX tablet described in the '940 patent consists of a hydrophilic active matrix core containing alfuzosin hydrochloride and two inert, functional layers (one swellable layer and one erodible layer) whose functions are to control the hydration and swelling rate of the core, and thereby slow down and linearize the dissolution of the drug. When the tablet comes into contact with gastric juices, it increases considerably in volume and thus remains in the stomach for a longer time. In this manner, most of the drug is absorbed in a controlled manner in the portion of the gastrointestinal tract having the highest capacity for absorption. The alfuzosin is released in zero order from the dosage form developed using this technology. However, the manufacture of multi-layered tablets by this technology involves special facilities, is time consuming, complex to produce, and consequently relatively expensive.

US2006147530 discloses a sustained release formulation of alfuzosin having a single functional layer that includes alfuzosin, one or more release retarding ingredients, and one or more pharmaceutically acceptable excipients. The release retarding ingredients include cellulose derivatives such as hydroxypropyl methyl cellulose (HPMC) 2208, in particular two high viscosity (apparent viscosity greater than 15000 cP) HPMCs METHOCEL K100M CR and METHOCEL K15M CR (both from Dow) are disclosed in the example formulations of US2006147530. US2006147530 discloses pharmacokinetic parameters, determined only under fed (non-fasted) conditions, for only one formulation comprising 24.29% (by weight of total dosage form) METHOCEL K100M CR. The ratios of each of the geometric means of Cₘₐₓ, AUC₀₋₂₄, and AUC_{0-INF} for the formulation compared to 10 mg XATRAL XL were between 80-125%.

The present invention addresses the need for improved extended release alfuzosin compositions, particularly extended release alfuzosin compositions that are bioequivalent to the current marketed dosage form at both non-fasted and fasted administration conditions.

### SUMMARY

Disclosed herein are improved alfuzosin compositions for extended release of alfuzosin. The alfuzosin compositions are useful in treating or preventing conditions such as signs and symptoms of benign prostatic hyperplasia, lower urinary tract symptoms, female sexual dysfunction, premature ejaculation, and primary dysmenorrheal and for managing blood pressure or delaying onset of male ejaculation.

In an embodiment, the alfuzosin composition comprises a tablet core comprising alfuzosin, and a release-retarding matrix, wherein the release-retarding matrix comprises about 40 to about 65 weight % hydroxypropyl methyl cellulose (HPMC) 2208 with a maximum apparent viscosity of about 5600 cP, based on the total weight of the tablet core; and an extended-release coating substantially surrounding the tablet core comprising a release-retarding coating material; wherein the alfuzosin composition administered under fed conditions is bioequivalent to a reference dosage form of New Drug Application (NDA) #021287 administered under fed conditions and wherein the alfuzosin composition administered under fasted conditions is bioequivalent to the reference dosage form administered under fasted conditions.

In an embodiment, the alfuzosin composition comprises a tablet core comprising alfuzosin, and a release-retarding matrix, wherein the release-retarding matrix comprises about 40 to about 65 weight % hydroxypropyl methyl cellulose (HPMC) 2208 with a maximum apparent viscosity of about 5600 cP, based on the total weight of the tablet core; and an extended-release coating substantially surrounding the tablet core comprising a release-retarding coating material; wherein after a single administration of the composition to a human, the composition provides an area under the plasma alfuzosin concentration curve from time 0 to time t (AUC₀₋ₜ) of about 37 to about 475 hr*ng/ml under fed conditions or about 30 to about 239 hr*ng/ml under fasting conditions; an area under the plasma alfuzosin concentration curve from time 0 to infinity (AUC_{0-∞}) of about 42 to about 492 hr*ng/ml under fed conditions or about 36 to about 252 hr*ng/ml under fasting conditions; and a maximum plasma alfuzosin concentration (Cₘₐₓ) of about 4.5 to about 40.0 ng/ml under fed conditions or about 2.8 to about 21.4 ng/ml under fasting conditions.

In an embodiment, the alfuzosin composition comprises a tablet core comprising alfuzosin, and a release-retarding matrix, wherein the release-retarding matrix comprises about 40 to about 65 weight % hydroxypropyl methyl cellulose (HPMC) 2208 with a maximum apparent viscosity of about 5600 cP, based on the total weight of the tablet core; and an extended-release coating substantially surrounding the tablet core comprising a release-retarding coating material; wherein about 10% to about 17% of the alfuzosin is dissolved in 1 hour; about 25% to about 40% of the alfuzosin is dissolved in 3 hours; about 50% to about 70% of the alfuzosin is dissolved in 8 hours; and no less than about 80% of the alfuzosin is dissolved in 23 hours, wherein dissolution is determined according to USP 28 <711> test apparatus 2 (paddle) with a 100 rpm paddle speed at 37 C using Sandwich sinkers in sequential dissolution media as follows: 0.01 M HCl at 0-1 hr, pH 4.5 acetate buffer at 2-3 hr, and pH 6.8 phosphate buffer at 9-23 hr.

Methods of making and using the alfuzosin compositions are also disclosed herein.

These and other embodiments, advantages and features of the present invention become clear when detailed description and examples are provided in subsequent sections.

### DETAILED DESCRIPTION

Disclosed herein are improved alfuzosin compositions. The alfuzosin compositions are useful in treating or preventing conditions such as signs and symptoms of benign prostatic hyperplasia, lower urinary tract symptoms, female sexual dysfunction, premature ejaculation, and primary dysmenorrheal. The alfuzosin compositions are also useful in managing blood pressure or delaying onset of male ejaculation. Processes of using the compositions are also disclosed.

The terms “a” and “an” do not denote a limitation of quantity, but rather denote the presence of at least one of the referenced item. The term “or” means “and/or”. The terms “comprising”, “having”, “including”, and “containing” are to be construed as open-ended terms (i.e., meaning “including, but not limited to”).

An “active agent” means a compound (for example, alfuzosin), element, or mixture that when administered to a patient, alone or in combination with another compound, element, or mixture, confers, directly or indirectly, a physiological effect on the patient. The indirect physiological effect may occur via a metabolite or other indirect mechanism. When the active agent is a compound, then salts, solvates (including hydrates) of the free compound or salt, crystalline forms, non-crystalline forms, and any polymorphs of the compound are contemplated herein. Compounds may contain one or more asymmetric elements such as stereogenic centers, stereogenic axes and the like, e.g., asymmetric carbon atoms, so that the compounds can exist in different stereoisomeric forms. These compounds can be, for example, racemates or optically active forms. For compounds with two or more asymmetric elements, these compounds can additionally be mixtures of diastereomers. For compounds having asymmetric centers, all optical isomers in pure form and mixtures thereof are encompassed. In addition, compounds with carbon-carbon double bonds may occur in Z- and E-forms, with all isomeric forms of the compounds. In these situations, the single enantiomers, i.e., optically active forms can be obtained by asymmetric synthesis, synthesis from optically pure precursors, or by resolution of the racemates. Resolution of the racemates can also be accomplished, for example, by conventional methods such as crystallization in the presence of a resolving agent, or chromatography, using, for example a chiral HPLC column. All forms are contemplated herein regardless of the methods used to obtain them.

“Alfuzosin” ((R,S)—N-[3-[(4-amino-6,7-dimethoxy-2-quinazolinyl)methylamino]propyl]tetrahydro-2-furancarboxamide) as used herein is inclusive of all pharmaceutically acceptable salt forms, crystalline forms, amorphous forms, polymorphic forms, solvates, and hydrates unless specifically indicated otherwise. As used herein, “Alfuzosin HCl” means (R,S)—N-[3-[(4-amino-6,7-dimethoxy-2-quinazolinyl)methylamino]propyl]tetrahydro-2-furancarboxamide hydrochloride unless otherwise indicated.

“Bioavailability” means the extent or rate at which an active agent is absorbed into a living system or is made available at the site of physiological activity. For active agents that are intended to be absorbed into the bloodstream, bioavailability data for a given formulation may provide an estimate of the relative fraction of the administered dose that is absorbed into the systemic circulation. “Bioavailability” can be characterized by one or more pharmacokinetic parameters.

“Bioequivalence” means the absence of a significant difference in the rate or extent to which the active agent in pharmaceutical equivalents or pharmaceutical alternatives is absorbed into a living system or is made available at the site of physiological activity. Bioequivalence can be determined by comparing in vitro dissolution testing data for a test dosage form and a reference dosage form or by comparing pharmacokinetic parameters for a test dosage form and a reference dosage form. For example, the comparison can be between an alfuzosin composition disclosed herein vs. the reference dosage form of NDA #021287.

A “dosage form” means a unit of administration of an active agent. Examples of dosage forms include tablets, capsules, injections, suspensions, liquids, emulsions, creams, ointments, suppositories, inhalable forms, transdermal forms, and the like.

The term “effective amount” or “therapeutically effective amount” means an amount effective, when administered to a patient, to provide any therapeutic benefit. A therapeutic benefit may be an amelioration of symptoms, e.g., an amount effective to decrease the symptoms of benign prostatic hyperplasia. The amount that is “effective” will vary from subject to subject, depending on the age and general condition of the individual, the particular active agent, and the like. Thus, it is not always possible to specify an exact “effective amount.” However, an appropriate “effective” amount in any individual case can be determined by one of ordinary skill in the art using routine experimentation. In certain circumstances a patient may not present symptoms of a condition for which the patient is being treated. A therapeutically effective amount of an active agent may also be an amount sufficient to provide a significant positive effect on any indicium of a disease, disorder, or condition, e.g. an amount sufficient to significantly reduce the severity of seizures. A significant effect on an indicium of a disease, disorder, or condition is statistically significant in a standard parametric test of statistical significance, for example Student's T-test, where p≦0.05. An “effective amount” or “therapeutically effective amount” of alfuzosin HCl can also be an amount of about 10 mg per day or less, or of any dosage amount approved by a governmental authority, such as the US FDA, for use in treatment. In some embodiments amounts of 10 mg alfuzosin HCl per day or 10 mg alfuzosin HCl per extended release once daily unit dosage form is an “effective amount” or “therapeutically effective amount” of alfuzosin HCl.

“Efficacy” means the ability of an active agent administered to a patient to produce a therapeutic effect in the patient.

As used herein “food” means a food with sufficient bulk and fat content that it is not rapidly dissolved and absorbed in the stomach. In one embodiment, the food is a meal, such as breakfast, lunch, or dinner. The terms “with food”, “fed” and “non-fasted” are equivalent. In one embodiment, a dosage of alfuzosin administered to a patient “with food” or in a a “non-fasted” (“fed”) state is administered to the patient starting between about 30 minutes prior to about 2 hours after eating a meal; more specifically, the dosage is administered within 30 minutes of starting to eat a meal. In another embodiment, with food means that the dosage form is administered at substantially the same time as the eating the meal.

The terms “without food”, “fasted”, or “an empty stomach” are equivalent. In one embodiment, fasted means the condition wherein no food is consumed within 1 hour prior to administration of the dosage form or 2 hours after administration of the dosage form. In another embodiment, fasted is defined to mean the condition of not having consumed food for about one hour prior to until about 2 hours after consumption of a dose.

As used herein, “immediate-release” means a dosage form in which greater than or equal to about 75% of the active ingredient is released within two hours, or, more specifically, within one hour, of administration. Immediate-release or controlled-release may also be characterized by their dissolution profiles.

“Oral dosage form” means a dosage form for oral administration.

As used herein, “alfuzosin therapy” refers to medical treatment of a symptom, disorder, or condition by administration of alfuzosin.

A “patient” means a human or non-human animal in need of medical treatment. Medical treatment can include treatment of an existing condition, such as a disease or disorder, prophylactic or preventative treatment, or diagnostic treatment. In some embodiments the patient is a human patient.

“Pharmacokinetic parameters” describe the in vivo characteristics of an active agent (or surrogate marker for the active agent) over time, such as plasma concentration (C), Cₘₐₓ, Cₙ, C₂₄, T_{max, and AUC}ₘₐₓ “Cₘₐₓ” is the measured concentration of the active agent in the plasma at the point of maximum concentration. “Cₙ” is the measured concentration of an active agent in the plasma at about n hours after administration. “C₂₄” is the measured concentration of an active agent in the plasma at about 24 hours after administration. The term “Tₘₐₓ” refers to the time at which the measured concentration of an active agent in the plasma is the highest after administration of the active agent. “AUC” is the area under the curve of a graph of the measured concentration of an active agent (typically plasma concentration) vs. time, measured from one time point to another time point. For example AUC₀₋ₜ, is the area under the curve of plasma concentration versus time from time 0 to time t. The AUC_{0-∞} or AUC_{0-INF} is the calculated area under the curve of plasma concentration versus time from time 0 extrapolated to time infinity. Pharmacokinetic parameters can be determined for alfuzosin.

A “product” or “pharmaceutical product” means a dosage form of an active agent plus published material, and optionally packaging.

“Reference dosage form” means an alfuzosin dosage form as described in FDA New Drug Application No. #021287 approved on Jun. 12, 2003.

“Reference listed drug” (RLD) under 21 CFR 314.94(a)(3) means the listed dosage form identified by the FDA as the dosage form upon which an applicant relies in seeking approval of its ANDA to market a generic equivalent dosage form as provided in the FDA's Approved Drug Products with Therapeutic Equivalence Evaluations (“Orange Book”). As of the filing date of this application, UROXATRAL® 10 mg is the RLD for the alfuzosin HCl dosage form described in FDA New Drug Application No. #021287.

“Safety” means the incidence or severity of adverse events associated with administration of an active agent, including adverse effects associated with patient-related factors (e.g., age, gender, ethnicity, race, target illness, abnormalities of renal or hepatic function, co-morbid illnesses, genetic characteristics such as metabolic status, or environment) and active agent-related factors (e.g., dose, plasma level, duration of exposure, or concomitant medication).

“Pharmaceutically acceptable salts” includes derivatives of alfuzosin, and other active agents, wherein the active agent is modified by making acid or base addition salts thereof, and further refers to pharmaceutically acceptable solvates, including hydrates, crystalline forms, non-crystalline forms, polymorphs, and stereoisomers of such compounds and such salts. Examples of pharmaceutically acceptable salts include mineral or organic acid addition salts of basic residues such as amines; alkali or organic addition salts of acidic residues; and the like, and combinations comprising one or more of the foregoing salts. The pharmaceutically acceptable salts include salts and the quaternary ammonium salts of the alfuzosin. For example, salts include those derived from inorganic acids such as hydrochloric, hydrobromic, sulfuric, sulfamic, phosphoric, nitric and the like; other acceptable inorganic salts include metal salts such as sodium salt, potassium salt, cesium salt, and the like; and alkaline earth metal salts, such as calcium salt, magnesium salt, and the like, and combinations comprising one or more of the foregoing salts. Pharmaceutically acceptable organic salts includes salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH2)n-COOH where n is 0-4, and the like; organic amine salts such as triethylamine salt, pyridine salt, picoline salt, ethanolamine salt, triethanolamine salt, dicyclohexylamine salt, N,N′ dibenzylethylenediamine salt, and the like; and amino acid salts such as arginate, asparginate, glutamate, and the like; and combinations comprising one or more of the foregoing salts.

Solid dosage forms of alfuzosin HCl comprise up to about 50 mg of alfuzosin HCl, specifically about 2.5 to about 50 mg of alfuzosin HCl, more specifically about 2.5 to about 20 mg alfuzosin HCl, yet more specifically about 10 mg alfuzosin HCl. In one embodiment, the solid dosage form is an oral dosage form, for example, a tablet. Specifically, the solid dosage form is an oral extended release tablet having 10 mg alfuzosin HCl for once daily dosage or an oral extended release tablet having 5 mg alfuzosin HCl for twice daily dosage.

“Extended-release” dosage forms include forms for which the release of the active agent is at such a rate that blood (e.g., plasma) levels are maintained within a therapeutic range for at least about 8 hours, specifically at least about 12 hours, and more specifically at least about 24 hours after administration at steady-state. The term steady-state in reference to a blood plasma level means that a plasma level for a given active agent has been achieved, and is maintained with subsequent doses of the active agent, which is at or above the minimum effective therapeutic level for the active agent.

The present invention relates to alfuzosin compositions comprising alfuzosin and a release retarding matrix. Specifically, the alfuzosin composition comprises alfuzosin and a release-retarding matrix comprising about 35 to about 80% (by weight of the total composition) hydroxypropyl methyl cellulose (HPMC). In some embodiments, the HPMC is HPMC 2208 with a maximum apparent viscosity of about 5600 cP. The alfuzosin composition can be in the form of a compressed extended release tablet. Such compositions were found, surprisingly, to be bioequivalent to the reference drug described in NDA #021287 at both non-fasting and fasting conditions. In certain embodiments, the composition was surprisingly observed to have a reduced food effect compared to the reference drug described in NDA #021287, i.e., the composition was observed to have improved bioavailability under fasting conditions as compared to the reference drug described in NDA #021287.

The release-retarding matrix comprises about 40 to about 70% (by weight of the total composition) HPMC 2208 with an apparent viscosity of about 3000 to about 5600 cP. In some embodiments, the release-retarding matrix further comprises about 10 to about 40% (by weight of the total composition) HPMC 2208 with an apparent viscosity of about 80 to about 120 cP or up to about 40% (by weight of the total composition) ethyl cellulose with an ethoxyl substitution greater than about 49.5%. In an embodiment, the release-retarding matrix comprises about 40% (by weight) hydroxypropyl methyl cellulose 2208 having a viscosity of 3000-5600 cP; about 10 to about 40% (by weight) hydroxypropyl methyl cellulose 2208 having a viscosity of 80-120 cP; and up to about 30% (by weight) ethyl cellulose with an ethoxyl substitution greater than about 49.5%. Apparent viscosity of the HPMC is determined using the USP <911> viscosity test for a 2% (by weight) aqueous solution at 20° C. using an Ubbelohde viscometer.

In some embodiments, the alfuzosin composition is bioequivalent to alfuzosin oral tablet dosage forms commercially available in the United States.

In an embodiment, bioequivalence of an alfuzosin composition to a reference dosage form is determined by an in vivo pharmacokinetic study to determine a pharmacokinetic parameter for the alfuzosin composition. Specifically, bioequivalence can be determined by an in vivo pharmacokinetic study to determine a pharmacokinetic parameter for the alfuzosin composition and the reference dosage form.

A pharmacokinetic parameter for the alfuzosin composition or the reference dosage form can be measured in a single or multiple dose pharmacokinetic study using a replicate or a nonreplicate design. For example, the pharmacokinetic parameters for an alfuzosin composition and for a reference dosage form can be measured in a single dose pharmacokinetic study using a two-period, two-sequence crossover design. Alternately, a four-period, replicate design crossover study may also be used. Single doses of the test composition and reference dosage form are administered and blood or plasma levels of the active agent or a surrogate marker for the active agent are measured over time. Pharmacokinetic parameters characterizing rate or extent of active agent absorption are evaluated statistically.

The area under the plasma concentration-time curve from time zero to the time of measurement of the last quantifiable concentration (AUC₀₋ₜ) and to infinity (AUC_{0-∞}), Cₘₐₓ, and Tₘₐₓ can be determined according to standard techniques. Statistical analysis of pharmacokinetic data is performed on logarithmic transformed data (e.g., AUC₀₋ₜ, AUC_{0-∞}, or Cₘₐₓ data) using analysis of variance (ANOVA).

In an embodiment, the alfuzosin compositions are bioequivalent to tablet formulations commercially available in the United States, for example UROXATRAL®, the reference dosage form of NDA #021287.

In one embodiment, bioequivalence means the composition has a geometric mean of logarithmic transformed Cₘₐₓ, AUC_{0-∞}, or AUC₀₋ₜ that is 50% to 200% of that of UROXATRAL®.

In one embodiment, alfuzosin is measured in an in vivo pharmacokinetic study to determine a pharmacokinetic parameter for the alfuzosin composition. The alfuzosin composition is determined to be bioequivalent to UROXATRAL® if it has a geometric mean of logarithmic transformed AUC₀₋ₜ, AUC_{0-INF}, or Cₘₐₓ for alfuzosin as described for an equivalent strength of UROXATRAL®. Specifically, the geometric mean AUC₀₋ₜ of alfuzosin for a 10 mg dosage form of UROXATRAL® is about 99 to about 512 hr*ng/ml under fed conditions or about 58 to about 379 hr*ng/ml under fasting conditions, the geometric mean AUC_{0-INF} of UROXATRAL® is about 106 to about 554 hr*ng/ml under fed conditions or about 68 to about 438 hr*ng/ml under fasting condition, and the geometric mean Cₘₐₓ of UROXATRAL® is about 7.1 to about 33.2 ng/ml under fed conditions or about 3.7 to about 18.0 ng/ml under fasting conditions.

In an embodiment, alfuzosin levels are measured for the alfuzosin composition in a fasting pharmacokinetic study. The geometric mean of logarithmic transformed AUC₀₋ₜ of alfuzosin is within about 70% to about 143%, specifically within about 80% to about 125% of about 136 hr*ng/ml; the geometric mean of logarithmic transformed AUC_{0-∞} of alfuzosin is within about 70% to about 143%, specifically within about 80% and about 125% of about 149 hr*ng/ml; or the geometric mean of logarithmic transformed Cₘₐₓ of alfuzosin is within about 70% and about 143%, specifically within about 80% and about 125% of about 7.9 ng/ml.

In an embodiment, alfuzosin levels are measured for the alfuzosin composition in a non-fasting (fed) pharmacokinetic study. The geometric mean of logarithmic transformed AUC₀₋ₜ of alfuzosin is within about 70% to about 143%, specifically within about 80% to about 125% of about 213 hr*ng/ml; the geometric mean of logarithmic transformed AUC_{0-∞} of alfuzosin is within about 70% to about 143%, specifically within about 80% and about 125% of about 228 hr*ng/ml; or the geometric mean of logarithmic transformed Cₘₐₓ of alfuzosin is within about 70% and about 143%, specifically within about 80% and about 125% of about 15.8 ng/ml.

In an embodiment, the alfuzosin composition is determined to be bioequivalent to a reference dosage form when the alfuzosin composition has a geometric mean of logarithmic transformed AUC₀₋ₜ, AUC_{0-∞}, or Cₘₐₓ for alfuzosin within about 70% and about 143%, more specifically within about 80% and about 125%, of the value of a geometric mean of logarithmic transformed AUC₀₋ₜ, AUC_{0-∞}, or Cₘₐₓ described for an equivalent strength of UROXATRAL® in NDA #021287.

In an embodiment, a pharmacokinetic study of the alfuzosin composition is performed under non-fasted or fasted conditions.

In an embodiment, the pharmacokinetic study is conducted between the alfuzosin composition and the reference dosage form using the strength specified by the FDA in APPROVED DRUG PRODUCTS WITH THERAPEUTIC EQUIVALENCE EVALUATIONS (ORANGE BOOK) as the RLD.

In some embodiments, bioequivalence is determined by performing an in vivo pharmacokinetic study to compare each strength of the alfuzosin composition with each corresponding strength of UROXATRAL®. In other embodiments, bioequivalence is determined by performing an in vivo pharmacokinetic study to compare the alfuzosin composition at the strength of the RLD for UROXATRAL® and at other strengths, bioequivalence is determined by performing dissolution testing to determine that the alfuzosin compositions have a dissolution profile that is substantially identical to the dissolution profile of their corresponding strength of UROXATRAL® determined using the same method and conditions.

A dissolution profile is data measuring the cumulative amount of active agent released from a dosage form as a function of time. A dissolution profile can be measured utilizing the USP 28 Drug Release Test <724>, which incorporates standard test USP 28 Test <711>. A dissolution profile is characterized by the test conditions selected such as, for example, apparatus type, shaft speed, temperature, volume, and pH of the dissolution medium. Dissolution profiles for a dosage form may be measured at more than one set of test conditions. For example, a first dissolution profile can be measured at a pH level approximating that of the stomach, and a second dissolution profile can be measured at a pH level approximating that of one point in the intestine or several pH levels approximating multiple points in the intestine.

With respect to two dissolution profiles, “substantially identical” means the difference at any dissolution sampling time point under the same testing conditions between the alfuzosin composition and the reference composition is not greater than 15% or if the dissolution profiles are compared using the following equation that defines a similarity factor (f₂), an f₂ value for the two dissolution profiles is between 50 and 100:

$${f}_{2} = 50 LOG \{ {[ 1 \div 1 / n {\sum }_{t - 1}^{n} {( {R}_{t} - {T}_{t} )}^{2} ]}^{- 0.5} \times 100 \}$$

where LOG=logarithm to base 10, n=number of sampling time points, Σ=summation over all time points, Rₜ=dissolution at time point t of the reference, and Tₜ=dissolution at time point t of the test (oxcarbazepine solution). (See for example FDA Guidance for Industry SUPAC-MR: Modified Release Solid Oral Dosage Forms, Scale-Up and Postapproval Changes: Chemistry, Manufacturing, and Controls; In Vitro Dissolution Testing and In Vivo Bioequivalence Documentation, September 1997, pp. 32-33).

The alfuzosin composition can have a dissolution profile that is substantially identical to the dissolution profile of an alfuzosin tablet dosage form commercially available in the United States determined using the same method in the same dissolution medium.

In an embodiment, the alfuzosin composition has a dissolution profile that is substantially identical to a dissolution profile of an equivalent strength of a reference dosage form of NDA #021287. Specifically, the alfuzosin composition can have a dissolution profile that is substantially identical to the dissolution profile of UROXATRAL®.

The dissolution profiles of the alfuzosin composition and a commercially available dosage form, for example UROXATRAL® of NDA #021287, are determined by in vitro dissolution testing at one or more strengths of each using the same test method and conditions. Dissolution profiles of the alfuzosin composition and a commercially available dosage form, for example UROXATRAL® of NDA #021287, can also be determined at multiple test conditions.

In some embodiments, the dissolution profile is determined using the conditions according to USP 28 <711> test method 2 (paddle). The dissolution profile is determined in a particular buffer, for example, in 0.01 M HCl in water, pH 4.5 acetate buffer, or pH 6.8 phosphate buffer.

Herein, the “alfuzosin dissolution test” means a dissolution test that is performed at the conditions according to USP 28 <711> test method 2 (paddle) using 500 mL of 0.01 M HCl in water a USP 2 apparatus with a 100 rpm paddle speed at 37 C. Under such test conditions, at least about 5% of the alfuzosin should be dissolved in 1 hour and at least about 80% of the alfuzosin is dissolved in 20 hours. Specifically, about 10 to about 17% of the alfuzosin is dissolved in 1 hour; about 20% to about 40% of the alfuzosin is dissolved in 3 hours; about 45% to about 70% of the alfuzosin is dissolved in 8 hours; and at least about 80 of the alfuzosin is dissolved in 24 hours.

Dissolution profiles determined using the conditions according to USP 28 <711> test method 2 (paddle) for pH 4.5 acetate buffer or pH 6.8 phosphate buffer are disclosed in Example 2.

In some embodiments, the dissolution profile is determined using the conditions according to USP 28 <711> test method 3 (reciprocating cylinder). The dissolution profile is determined in a particular buffer, for example, in 0.01 M HCl in water (pH 1.2), pH 4.5 acetate buffer, or pH 6.8 phosphate buffer.

Herein, the “BioDisc dissolution test” means a dissolution test that is performed at the conditions according to USP 28 <711> test method 3 (reciprocating cylinder) using 250 mL of 0.01N HCl (0-1 hr), Acetate Buffer pH 4.5 (2-3 hr) and pH 6.8 phosphate Buffer (9-23 hr) at 37 C. Under such test conditions, at least about 10% of the alfuzosin should be dissolved in 1 hour and at least about 80% of the alfuzosin is dissolved in 23 hours. Specifically, about 10 to about 20% of the alfuzosin is dissolved in 1 hour; about 24% to about 40% of the alfuzosin is dissolved in 3 hours; about 51% to about 63% of the alfuzosin is dissolved in 9 hours; about 73% to about 89% of the alfuzosin is dissolved in 16 hours; and at least about 95 of the alfuzosin is dissolved in 23 hours.

Solid dosage forms for oral administration include, but are not limited to, capsules, tablets, pills, powders, and granules. In such solid dosage forms, the active agent may be admixed with one or more of the following: (a) one or more inert excipients (or carriers), such as sodium citrate or dicalcium phosphate; (b) fillers or extenders, such as starches, lactose, sucrose, glucose, mannitol, and silicic acid; (c) binders, such as carboxymethylcellulose, alignates, gelatin, polyvinylpyrrolidone, sucrose, and acacia; (d) humectants, such as glycerol; (e) disintegrating agents, such as agar-agar, calcium carbonate, potato or tapioca starch, alginic acid, certain complex silicates, and sodium carbonate; (f) solution retarders, such as paraffin; (g) absorption accelerators, such as quaternary ammonium compounds; (h) wetting agents, such as cetyl alcohol and glycerol monostearate; (i) adsorbents, such as kaolin and bentonite; and (j) lubricants, such as talc, calcium stearate, magnesium stearate, solid polyethylene glycols, sodium lauryl sulfate, and combinations comprising one or more of the foregoing additives. For capsules, tablets, and pills, the dosage forms may also comprise buffering agents.

An extended-release dosage form comprises a release-retarding material. The release-retarding material can be, for example, in the form of a matrix or a coating. The release-retarding material is a material that permits release of the active agent at a sustained rate in an aqueous medium. The release-retarding material can be selectively chosen so as to achieve, in combination with the other stated properties, a desired in vitro release rate

In embodiments disclosed herein, the release-retarding matrix of the alfuzosin compositions comprises about 40 to about 80% by weight of the total composition hydroxypropyl methyl cellulose (HPMC) 2208 with a maximum apparent viscosity of about 5600 cP.

HPMC is pharmaceutically acceptable and non-ionic, such that no interactions between the polymer and other constituents are to be expected. HPMC provides a hydrophilic matrix in which the active agent is distributed uniformly, and is released over a sustained period of time.

The various HPMC quality grades commercially available differ in their molecular weight, which is correlated with the apparent viscosity of the HPMC. The molecular weight of the polymer used and its concentration in the tablet are of particular importance for the release of the active agent. Adjustment of these parameters to achieve desired results is possible. A higher molecular weight leads to an increase in gel strength and increased viscosity, reducing the release of the active agent due to a greater barrier to diffusion and slower erosion of the tablet. Increasing the polymer concentration in the preparation, i.e., the ratio HPMC/active agent, results in an increase of gel viscosity on the surface of the tablets. This delays the release of active agent from the gel layer. The concentration effect is, however, of limited relevance for HPMC grades of high molecular weight.

Low viscosity HPMC, i.e. HPMC with apparent viscosity of no more than about 5600 cP, was unexpectedly found to provide the desired in vitro dissolution profiles and to provide in vivo bioequivalence to commercially marketed alfuzosin extended release tablets, i.e. UROXATRAL® 10 mg, under both fed (non-fasted) and fasting conditions.

In an embodiment, the release-retarding matrix comprises about 40% to −about 70% HPMC 2208 with an apparent viscosity of about 3000 to about 5600 cP. One commercially available example is METHOCEL K4M CR, but the invention is not limited thereto. In some embodiments, the release-retarding matrix further comprises about 10 to about 40% HPMC 2208 with an apparent viscosity of about 80 to about 120 cP. One commercially available example is METHOCEL K100LV CR, but the invention is not limited thereto. Herein, the percentages represent weight % of the release-retarding material based on the total weight of the oral dosage form.

The release-retarding matrix of the alfuzosin compositions may further comprise additional release-retarding materials.

In an embodiment, the release-retarding matrix further comprises up to about 40% ethyl cellulose. In some embodiments, the ethyl cellulose is a high ethoxyl substituted ethyl cellulose, with an ethoxyl substitution greater than about 49.5%. One commercially available example is AQUALON T10 ethyl cellulose, but the invention is not limited thereto.

Other release-retarding materials can be hydrophilic and/or hydrophobic polymers. Release-retarding materials include, for example acrylic polymers, other alkylcelluloses, shellac, zein, hydrogenated vegetable oil, hydrogenated castor oil, and combinations comprising one or more of the foregoing materials. Suitable acrylic polymers include, for example, acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methacrylic acid anhydride), methyl methacrylate, polymethacrylate, poly(methyl methacrylate) copolymer, polyacrylamide, aminoalkyl methacrylate copolymer, glycidyl methacrylate copolymers, and combinations comprising one or more of the foregoing polymers. The acrylic polymer may comprise a methacrylate copolymers described in NF XXIV as fully polymerized copolymers of acrylic and methacrylic acid esters with a low content of quaternary ammonium groups.

Suitable alkylcelluloses include, for example, ethylcellulose. Those skilled in the art will appreciate that other cellulosic polymers, including other alkyl cellulosic polymers, can be substituted for part or all of the ethylcellulose.

Suitable hydrophilic polymers for use as a release retarding material are biocompatible. They are slowly soluble and/or slowly gellable and/or swell rapidly or at a different rate in aqueous liquids and then may optionally be broken down. Preferred polymers include hydroxymethylcellulose, hydroxyethylcellulose, hydroxypropylmethylcellulose (HPMC) having a molecular weight of from 1000 to 4,000,000, hydroxypropylcellulose having a molecular weight of from 2000 to 2,000,000, carboxyvinyl polymers, chitosans, mannans, galactomannans, xanthans, carrageenans, amylose, alginic acid, its salts and its derivatives, pectins, acrylates, methacrylates, acrylic/methacrylic copolymers, polyanhydrides, polyamino acids, poly(methyl vinyl ether/maleic anhydride) polymers, polyvinyl alcohols, glucans, scleroglucans, carboxymethylcellulose and its derivatives, ethylcellulose, methylcellulose and, in general, hydrophilic cellulose derivatives.

The composition can also include additives additional to the active agent and the release-retarding materials.

In some embodiments, the alfuzosin composition is expected have increased bioavailability under fasted administration conditions and to have a reduced food effect. In an embodiment, the composition is expected to be bioequivalent to UROXATRAL under non-fasted conditions, wherein bioequivalent means that the ratio of the geometric mean of a pharmacokinetic parameter under fasted conditions to the geometric mean of the parameter measured under non-fasted conditions is expected to be within a range of 0.80 to 1.25, specifically the ratio of the geometric mean of each of AUC₀₋ₜ, AUC_{0-∞}, and Cₘₐₓ under fasted conditions to the geometric mean of the parameter measured under non-fasted conditions is expected to be within a range of 0.80 to 1.25, more specifically the 90% confidence limits of the ratio of each parameter is expected to be within a range of 0.80 to 1.25; while showing an increased Cₘₐₓ as compared to UROXATRAL under fasted conditions, i.e., the ratio under fasted conditions of the geometric mean of Cₘₐₓ for the coated formulation to UROXATRAL is expected to be greater than 1.25, specifically greater than about 1.35, more specifically greater than about 1.5, and yet more specifically equal to about 2.0.

In another embodiment, a 10 mg alfuzosin composition exhibits a geometric mean of logarithmic transformed Cₘₐₓ within about 0.80 and about 1.25 of 14.2 ng/ml under fed conditions and exhibits a ratio of geometric mean of logarithmic transformed Cₘₐₓ for the composition to geometric mean of logarithmic transformed Cₘₐₓ for a reference dosage form of NDA #021287 greater than about 1.25 under fasting conditions, specifically greater than about 1.35, more specifically greater than about 1.5, and yet more specifically equal to about 2.0.

In some embodiments, the alfuzosin compositions comprising alfusozin and the release retarding matrix when coated with a porous polymer coat (“coated formulation”) can have desirable properties, for example, desirable in vitro dissolution properties or in vivo pharmacokinetic properties. The coated formulation can comprise a tablet core and an extended-release coating. In one embodiment, the coated formulation comprises a tablet core comprising alfuzosin or a pharmaceutically acceptable salt thereof and a release-retarding matrix comprising about 40 to about 65 weight %, based on the total weight of the tablet core, hydroxypropyl methyl cellulose (HPMC) 2208 with a maximum apparent viscosity of about 5600 cP; and an extended-release coating substantially surrounding the tablet core comprising a release-retarding coating material. Substantially surrounding the tablet core means that the coating coats at least about 75% of the surface of the core, specifically at least about 80% of the core surface, more specifically at least 90% of the core surface, yet more specifically at least 95% of the core surface, even yet more specifically at least 99% if the core surface. In some embodiments the coating completely coats the tablet core (i.e., 100% of the core surface). Methods of determining the percent of the core surface coated are known to those of skill in the art.

In an embodiment, a 10 mg alfuzosin coated formulation is expected to be bioequivalent under fasted and non-fasted conditions, wherein bioequivalent means that the ratio of the geometric mean of a pharmacokinetic parameter under fasted conditions to the geometric mean of the parameter measured under non-fasted conditions is expected to be within a range of 0.80 to 1.25, specifically the ratio of the geometric mean of each of AUC₀₋ₜ, AUC_{0-∞}, and Cₘₐₓ under fasted conditions to the geometric mean of the parameter measured under non-fasted conditions is expected to be within a range of 0.80 to 1.25, more specifically the 90% confidence limits of the ratio of each parameter is expected to be within a range of 0.80 to 1.25.

In some embodiments, the coated formulation is expected have increased bioavailability under fasted administration conditions and to have a reduced food effect. In an embodiment, the coated formulation is expected to be bioequivalent to UROXATRAL under non-fasted conditions, wherein bioequivalent means that the ratio of the geometric mean of a pharmacokinetic parameter under fasted conditions to the geometric mean of the parameter measured under non-fasted conditions is expected to be within a range of 0.80 to 1.25, specifically the ratio of the geometric mean of each of AUC₀₋ₜ, AUC_{0-∞}, and Cₘₐₓ under fasted conditions to the geometric mean of the parameter measured under non-fasted conditions is expected to be within a range of 0.80 to 1.25, more specifically the 90% confidence limits of the ratio of each parameter is expected to be within a range of 0.80 to 1.25; while showing an increased Cₘₐₓ as compared to UROXATRAL under fasted conditions, i.e., the ratio under fasted conditions of the geometric mean of Cₘₐₓ for the coated formulation to UROXATRAL is expected to be greater than 1.25, specifically greater than about 1.35, more specifically greater than about 1.5, and yet more specifically equal to about 2.0.

In another embodiment, a 10 mg alfuzosin coated formulation exhibits under fed conditions a geometric mean of logarithmic transformed Cₘₐₓ within about 0.80 and about 1.25 of 14.2 ng/ml and exhibits a ratio under fasting conditions of geometric mean of logarithmic transformed Cₘₐₓ for the composition to geometric mean of logarithmic transformed Cₘₐₓ for a reference dosage form of NDA #021287 greater than about 1.25, specifically greater than about 1.35, more specifically greater than about 1.5, and yet more specifically equal to about 2.0.

The tablet core comprising alfuzosin, or a pharmaceutically acceptable salt thereof, and a release-retarding matrix comprising about 40 to about 65 weight % hydroxypropyl methyl cellulose (HPMC) 2208 with a maximum apparent viscosity of about 5600 cP, based on the total weight of the tablet core can be prepared as described herein.

Once the core is formed, it is coated with an extended-release coating. The extended-release coating that substantially surrounds the core comprises a release-retarding coating material and optional other components, such as plasticizers, pore formers, and the like.

The extended-release coating is present in the formulation at about 0.1 to about 30 wt. % based on the total weight of the core and the extended-release coating, specifically about 1.0 to about 25 wt. %, more specifically about 2.0 to about 20 wt. %, and yet more specifically about 5.0 to about 15 wt. %.

The extended-release coating is provided on the core using known coating processes such as simple or complex coacervation, interfacial polymerization, liquid drying, thermal and ionic gelation, spray drying, spray chilling, fluidized bed coating, pan coating, electrostatic deposition, compression coating, dry polymer powder coating, and the like.

The release-retarding coating material is, for example, in the form of a film coating comprising a dispersion of a hydrophobic polymer. Solvents used for application of the controlled-release coating include pharmaceutically acceptable solvents, such as water, methanol, ethanol, acetone, methylene chloride, and a combination comprising at least one of the foregoing solvents.

The extended-release profile of the active agent (either in vivo or in vitro) can be altered, for example, by using more than one release-retarding coating material, varying the thickness of the release-retarding coating material, changing the particular release-retarding coating material used, altering the relative amounts of release-retarding coating material, use of a plasticizer, altering the manner in which the plasticizer is added (e.g., when the extended-release coating is derived from an aqueous dispersion of hydrophobic polymer), by varying the amount of plasticizer relative to release-retarding coating material, by the inclusion of an additional coating excipient, by altering the method of manufacture, and the like.

Exemplary release-retarding coating materials include film-forming polymers such as an alkylcellulose including methylcellulose or ethylcellulose, a hydroxyalkylcellulose such as hydroxymethylcellulose, hydroxyethylcellulose, hydroxypropylcellulose and hydroxybutylcellulose, a hydroxyalkyl alkylcellulose such as hydroxyethyl methylcellulose and hydroxypropyl methylcellulose, a carboxyalkylcellulose such as carboxymethylcellulose, an alkali metal salt of carboxyalkylcelluloses such as sodium carboxymethylcellulose, a carboxyalkyl alkylcellulose such as carboxymethyl ethylcellulose, a carboxyalkylcellulose ester, a starch, a pectin such as sodium carboxymethylamylopectine, a chitin derivate such as chitosan, a polysaccharide such as alginic acid, alkali metal and ammonium salts thereof, a carrageenan, a galactomannan, traganth, agar-agar, gum arabicum, guar gum and xanthan gum, a polyacrylic acid and the salts thereof, a polymethacrylic acid and the salts thereof, a methacrylate copolymer, a polyvinylalcohol, a polyvinylpyrrolidone, a copolymer of polyvinylpyrrolidone with vinyl acetate, a polyalkylene oxide such as polyethylene oxide and polypropylene oxide and a copolymer of ethylene oxide and propylene oxide, or a combination comprising at least one of the foregoing materials.

The extended-release coating optionally comprises a plasticizer, an additional film-former, a pore former, or a combination comprising at least one of the foregoing materials.

The formulations optionally further comprises a non-functional coating. By “functional coating” is meant to include a coating that modifies the release properties of the total formulation, for example, a controlled-release coating that provides sustained-release. By “non-functional coating” is meant to include a coating that does not significantly modify the release properties of the total formulation, for example, a cosmetic coating or an interlayer coating used to separate a functional coating from other components of the formulation. A non-functional coating can have some impact on the release of the active agent due to the initial dissolution, hydration, perforation of the coating, and the like, but would not be considered to be a significant deviation from the non-coated composition.

A pore forming material is optionally added to the controlled-release coating to promote release of the active agent from the core. The pore forming material is organic or inorganic; it is a material that can be dissolved, extracted or leached from the coating in the environment of use; or it can have a pH-dependent solubility property; and the like. Exemplary pore forming materials include hydrophilic polymers such as a hydroxy alkyl-alkyl cellulose (e.g., hydroxypropylmethyl cellulose, and the like), a hydroxyl alkyl cellulose (e.g., hydroxypropylcellulose, and the like), or a povidone; a saccharide (e.g., lactose, and the like); an inorganic salt (e.g., dibasic calcium phosphate, sodium chloride, and the like); a polyethylene glycol (e.g., polyethylene glycol (PEG) 1450, and the like); a sugar alcohol (e.g., sorbitol, mannitol, and the like); an alkali alkyl sulfate (e.g., sodium lauryl sulfate); a polyoxyethylene sorbitan fatty acid ester (e.g., polysorbate); methyacrylate copolymers; or a combination comprising at least one of the foregoing pore forming materials.

A specific release-retarding coating material includes ethyl cellulose and optionally in combination with hydroxypropymethyl cellulose.

In one embodiment, the ratio of ethyl cellulose to hydroxypropylmethyl cellulose is about 90:10, specifically about 70:30, more specifically about 60:40, and yet more specifically about 50:50.

Further, it has surprisingly been found that in some embodiments, co-compressed alfuzosin tablets, for example, “tablet-in-tablet” dosage forms, may have desirable properties, for example, desirable in vitro dissolution properties or in vivo pharmacokinetic properties. The co-compressed tablets can comprise a core and an outer compressed coat. In one embodiment, a co-compressed tablet comprises a core comprising alfuzosin and a binder; and an outer coat comprising alfuzosin and a release retarding matrix; wherein the outer coat covers the core from all sides. In one embodiment, the co-compressed tablet is bioequivalent to a reference dosage form of NDA #021287.

The core of the co-compressed tablet can be an immediate release core. The binder in the core can be a water insoluble polymer. Suitable water insoluble polymers include, but are not limited to, microcrystalline cellulose, ethyl cellulose, cellulose acylate, cellulose diacylate, cellulose triacylate, cellulose monoacetate, cellulose diacetate, cellulose triacetate, sodium carboxymethyl cellulose, monocellulose alkanylate, dicellulose alkanylate, tricellulose alkanylate, monocellulose alkenylates, dicellulose alkenylates, tricellulose alkenylates, monocellulose aroylates, dicellulose aroylates, tricellulose aroylates, or a combination comprising two or more of the foregoing polymers. In one embodiment, the water insoluble polymer is microcrystalline cellulose, for example, Avicel® PH 101 and Avicel® PH 102.

The core of the co-compressed tablet may further optionally comprise a filler, a disintegrant, a lubricant, or a combination comprising two or more of the foregoing excipients.

The core of the co-compressed tablet is present in an amount of about 5 wt % to about 30 wt %, or more specifically, 10 wt % to 20 wt %, or even more specifically, about 16 wt %, based on the total weight of the co-compressed tablet.

The alfuzosin in the core of the co-compressed tablet is present in an amount of about 0.2 wt % to about 1.0 wt %, or more specifically, 0.5 wt % to 0.7 wt %, or even more specifically, about 0.57 wt %, based on the total weight of the co-compressed tablet.

The release retarding matrix in the outer coat of the co-compressed tablet can comprise a release retarding material that can be a cellulosic polymer, a polyvinyl acetate, a polymethacrylate, a water soluble polymer, or a combination comprising at least one of the foregoing release retarding materials. In one embodiment, the release retarding material is a combination comprising hydroxypropyl methyl cellulose (HPMC) with an apparent viscosity of about 3,000 to about 20,000 cps and ethyl cellulose. As used herein, the term “apparent viscosity” of an HPMC means the viscosity of 2% (w/v) aqueous solution of the HPMC in water measured at 20° C. HPMCs vary in the chain length of their cellulosic backbone and consequently in their apparent viscosities. HPMC used herein can have an apparent viscosity of about 3000 to about 20,000 cps, or more specifically, about 3,000 to about 5,600 cps, or even more specifically, about 5,600 cps.

The outer coat of the co-compressed tablet may further optionally comprise additional excipients, for example, a filler, a binder, a disintegrant, a lubricant, a glidant, or a combination comprising two or more of the foregoing excipients. In one embodiment, the outer coat further comprises hydroxypropyl methyl cellulose with an apparent viscosity of about 80 to about 120 cps, magnesium stearate, and colloidal silicon dioxide.

The outer coat of the co-compressed tablet is present in an amount of about 70 wt % to about 95 wt %, or more specifically, 80 wt % to 90 wt %, or even more specifically, about 84 wt %, based on the total weight of the co-compressed tablet.

The alfuzosin in the outer coat of the co-compressed tablet is present in an amount of about 0.5 wt % to about 3.0 wt %, or more specifically, 1.5 wt % to 2.5 wt %, or even more specifically, about 2.28 wt %, based on the total weight of the co-compressed tablet.

In another embodiment, a co-compressed tablet comprises a core consisting essentially of alfuzosin and a binder; and an outer coat consisting essentially of alfuzosin and a release retarding matrix; wherein the outer coat covers the core from all sides. In one embodiment, the co-compressed tablet is bioequivalent to a reference dosage form of NDA #021287. In one embodiment, the binder in the core is microcrystalline cellulose, for example, Avicel® PH 101 or Avicel® PH 102. In another embodiment, the release retarding matrix in the outer coat consists essentially of hydroxypropyl methyl cellulose with an apparent viscosity of about 3000 to about 20000 cps and ethyl cellulose. In yet another embodiment, the release retarding matrix in the outer coat further comprises hydroxypropyl methyl cellulose with an apparent viscosity of about 80 to about 120 cps. In still another embodiment, the outer coat further comprises a lubricant that is a combination consisting essentially of magnesium stearate and colloidal silicon dioxide.

In yet another embodiment, a co-compressed tablet comprises a core consisting essentially of alfuzosin and microcrystalline cellulose; and an outer coat consisting essentially of alfuzosin, hydroxypropyl methyl cellulose with an apparent viscosity of about 3000 to about 20000 cps, ethyl cellulose, hydroxypropyl methyl cellulose with an apparent viscosity of about 80 to about 120 cps, magnesium stearate, and colloidal silicon dioxide; wherein the outer coat covers the core from all sides. In one embodiment, the co-compressed tablet is bioequivalent to a reference dosage form of NDA #021287.

In some other embodiments, the co-compressed tablets described above exhibit a dissolution profile that is substantially identical to a dissolution profile of a reference dosage form of NDA #021287.

In some other embodiments, the co-compressed tablets described above exhibit a dissolution profile such that about 10 to about 17% of the alfuzosin is released in 1 hour; about 20% to about 40% of the alfuzosin is released in 3 hours; about 45% to about 70% of the alfuzosin is released in 8 hours; and at least about 80% of the alfuzosin is released in 24 hours; after combining the co-compressed tablets with 500 ml of 0.01 N HCl at 37° C. using USP Type II Apparatus (USP28, <711> Dissolution), at a paddle speed of 100 rpm. In other embodiments, the dissolution medium is pH 4.5 acetate buffer or a pH 6.8 phosphate buffer. In an embodiment, a method of making a co-compressed tablet, wherein the co-compressed tablet comprises a core and an outer coat, comprises mixing alfuzosin with a binder to form a core blend; compressing the core blend to form a core; mixing alfuzosin with a release retarding material and an optional lubricant to form an outer coat blend; adding about 50 wt % of the outer coat blend, based on the total weight of the outer coat of the co-compressed tablet, to a die cavity; adding the core to the die cavity; adding about 50 wt % of the outer coat blend, based on the total weight of the outer coat of the co-compressed tablet, to the die cavity; and compressing the content in the die to form the co-compressed tablet. In one embodiment, the co-compressed tablet obtained using the above method is bioequivalent to a reference dosage form of NDA #021287. In another embodiment, the co-compressed tablet obtained using the above method exhibits a dissolution profile that is substantially identical to a dissolution profile of a reference dosage form of NDA #021287, specifically the dissolution profile in the alfuzosin dissolution test is substantially identical to the dissolution profile of a reference dosage form of NDA #021287.

In one embodiment, the alfuzosin and the binder of the core of the co-compressed tablet were sized before being mixed to form the core blend. Suitable sizing operations include milling or sieving. Milling is often used to reduce the particle size of solid materials. Many types of mills are available and one of the most commonly used types of mill is the hammer mill. Alternatively, manual sieving through suitable sized mesh screens can be used to reduce particle sizes. In one embodiment, a # 20 mesh screen was used to reduce particle sizes.

In another embodiment, the alfuzosin and the release retarding material of the outer coat were screened before being mixed to form the outer coat blend. In one embodiment, the screening was conducted using a # 20 mesh screen.

In another embodiment, the optional lubricant of the outer coat was screened before being mixed to form the outer coat blend. In another embodiment, the screening was conducted using a # 30 mesh screen.

In yet another embodiment, the co-compressed tablets have a hardness of about 1.5 to about 50 kp, or more specifically, about 10 to about 30 kp, or even more specifically, about 20 kp.

The co-compressed tablets can optionally be further coated with an additional coating. The additional coating may be any suitable coating, such as, for example, a functional or a non-functional coating, or multiple functional and/or non-functional coatings. In one embodiment, the co-compressed tablets are optionally coated with an additional non-functional cosmetic coating.

The alfuzosin compositions are useful in treating or preventing conditions such as signs and symptoms of benign prostatic hyperplasia, lower urinary tract symptoms, female sexual dysfunction, premature ejaculation, and primary dysmenorrheal and for managing blood pressure or delaying onset of male ejaculation.

Methods of treatment with the alfuzosin compositions are provided herein. Such methods include administering the alfuzosin composition to a patient. The patient may be in need of alfuzosin therapy. Methods of treatment may also include providing the patient with the alfuzosin composition.

The following examples further illustrate the invention but, of course, should not be construed as in any way limiting its scope.

### EXAMPLES

### Example 1

### Alfuzosin Extended Release Formulations

The extended release formulations of this example comprise alfuzosin HCl as follows:

TABLE 1
Extended release formulations of alfuzosin HCl

| | | | | | | | BB5690240/ | |
|---|---|---|---|---|---|---|---|---|
| | BB5700195 | | BB5700198 | | NB1387:36 | | NB1387:39 | |
| | I | | II | | III | | IV | |
| Ingredients | Mg/tablet | % | Mg/tablet | % | Mg/tablet | % | Mg/tablet | % |
| 1 Alfuzosin Hydrochloride | 10 | 2.86 | 10 | 2.86 | 10 | 2.86 | 10 | 2.86 |
| 2 HPMC K4MCR (Dow) | 280 | 80.00 | 140 | 40.00 | 140 | 40 | 140 | 40 |
| 3 Ethyl Cellulose T10 | 0 | 0 | 140 | 40.00 | 70 | 20 | 0 | 0 |
| 4 HPMC K100LV (Dow) | 0 | 0 | 0 | 0 | 70 | 20 | 140 | 40 |
| 5 Avicel pH 101 | 54 | 15.43 | 54 | 15.43 | 54 | 15.43 | 54 | 15.43 |
| 6 Magnesium Stearate | 4 | 1.14 | 4 | 1.14 | 4 | 1.14 | 4 | 1.14 |
| 7 Colloidal silicon dioxide, NF | 2 | 0.57 | 2 | 0.57 | 2 | 057 | 2 | 0.57 |
| Total | 350 | 100.00 | 350 | 100.00 | 350 | 100 | 100 | 100.00 |
| | | | | | | | BB5700239/ | |
| | NB1387:37 | | NB1387:38 | | NB1387:41 | | NB1387:42 | |
| | V | | VI | | VII | | VIII | |
| Ingredients | Mg/tablet | % | Mg/tablet | % | Mg/tablet | % | Mg/tablet | % |
| 1 Alfuzosin Hydrochloride | 10 | 2.86 | 10 | 2.86 | 10 | 2.86 | 10 | 2.86 |
| 2 HPMC K4MCR (Dow) | 140 | 40.00 | 140 | 40.00 | 175 | 50 | 175 | 50 |
| 3 Ethyl Cellulose T10 | 87.5 | 25.00 | 105 | 30.00 | 0 | 0 | 70 | 20 |
| 4 HPMC K100LV (Dow) | 52.5 | 15.00 | 35 | 10.00 | 105 | 30 | 35 | 10 |
| 5 Avicel pH 101 | 54 | 15.43 | 54 | 15.43 | 54 | 15.43 | 54 | 15.43 |
| 6 Magnesium Stearate | 4 | 1.14 | 4 | 1.14 | 4 | 1.14 | 4 | 1.14 |
| 7 Colloidal silicon dioxide, NF | 2 | 0.57 | 2 | 0.57 | 2 | 057 | 2 | 0.57 |
| Total | 350 | 100.00 | 350 | 100.00 | 350 | 100.00 | 350 | 100.00 |

Alfuzosin Hydrochloride, HPMC and Ethyl Cellulose T10 (where applicable) were charged in a Gral 10 and mixed for 2 minutes at low speed and chopper off to form a mixture. The mixture was discharged and screened with a #20 mesh screen into a suitable container.

The screened mixture was charged into a Collette. Avicel screened with a #20 mesh screen was charged into the same Collette. The ingredients were mixed for 2 minutes at low speed and chopper off to form a second mixture.

Magnesium stearate and colloidal silicon dioxide were screened through a #30 mesh screen and charged into the Collette containing the second mixture. All ingredients were mixed for 1 minute at low speed and chopper off to form the final blend and then discharged into a suitable container.

The final blend was compressed into tablets on a Manesty using 12/32″ FFBE tooling to form the core tablets. Samples were taken for dissolution testing.

### Example 2

### Dissolution of Example 1 Alfuzosin Formulations I and II and UROXATRAL® Using USP 2 Apparatus

Dissolution of the Example 1 alfuzosin formulations I and II and UROXATRAL® was measured in 500 mL of 0.01 M HCl in water a USP 2 apparatus with a 100 rpm paddle speed at 37 C.

TABLE 2
Dissolution Profiles for Formulations I-II and UROXATRAL in 0.01 M HCl

| | Formulation I | | | | Formulation II | | | | UROXATRAL | | | |
|---|---|---|---|---|---|---|---|---|---|---|---|---|
| Time (hr) | mean | rsd | min | max | mean | rsd | min | max | mean | rsd | min | max |
| 1 | 12 | 13 | 10 | 17 | 16 | 7.3 | 15 | 19 | 16 | 9.3 | 14 | 18 |
| 2 | 19 | 13 | 16 | 26 | 25 | 6.3 | 23 | 29 | 23 | 9.3 | 20 | 26 |
| 3 | 25 | 12 | 22 | 34 | 32 | 5.8 | 29 | 37 | 28 | 9.4 | 25 | 32 |
| 4 | 31 | 12 | 27 | 41 | 38 | 5.5 | 35 | 43 | 33 | 9.4 | 29 | 38 |
| 6 | 41 | 11 | 35 | 53 | 49 | 6.4 | 45 | 55 | 43 | 9.5 | 37 | 50 |
| 8 | 49 | 10 | 43 | 63 | 58 | 6.3 | 53 | 66 | 54 | 10.1 | 46 | 63 |
| 12 | 64 | 9.1 | 56 | 78 | 73 | 5.6 | 67 | 82 | 74 | 11.5 | 64 | 90 |
| 16 | 77 | 7.7 | 68 | 90 | 83 | 4.5 | 78 | 91 | 89 | 7 | 82 | 100 |
| 20 | 87 | 6.9 | 77 | 100 | 91 | 3.6 | 86 | 97 | 98 | 2.9 | 95 | 103 |
| 24 | 94 | 5.9 | 85 | 104 | 97 | 2.8 | 92 | 101 | 103 | 1.3 | 101 | 105 |

Dissolution of the Example 1 alfuzosin formulations III and IV was also compared to dissolution of UROXATRAL® at the same conditions, as shown below in Table 3.

TABLE 3
Dissolution Profiles for Formulations III-IV and
VIII in 0.01 M HCl

| | | | | | | | | | Formulation |
|---|---|---|---|---|---|---|---|---|---|
| Time | Formulation III | | | | Formulation IV | | | | VIII |
| (hr) | mean | rsd | min | max | mean | rsd | min | max | mean |
| 1 | 15 | 5.1 | 15 | 17 | 13 | 4.2 | 12 | 14 | 14 |
| 2 | 24 | 5.9 | 22 | 26 | 21 | 4.2 | 20 | 22 | 22 |
| 3 | 31 | 5.9 | 30 | 35 | 28 | 5.0 | 27 | 31 | 29 |
| 4 | 38 | 5.8 | 36 | 42 | 35 | 5.6 | 33 | 39 | 34 |
| 6 | 51 | 6.2 | 48 | 55 | 48 | 6.5 | 43 | 52 | 45 |
| 8 | 62 | 6.9 | 57 | 67 | 60 | 7.8 | 54 | 68 | 54 |
| 12 | 80 | 7.2 | 74 | 86 | 77 | 5.9 | 72 | 85 | 69 |
| 16 | 92 | 5.7 | 87 | 98 | 92 | 5.1 | 88 | 101 | 82 |
| 20 | 101 | 2.9 | 97 | 104 | 102 | 3.3 | 99 | 107 | 93 |
| 24 | 106 | 1.1 | 104 | 107 | 102 | 2.6 | 98 | 105 | 99 |

Additionally, dissolution of the Example 1 alfuzosin formulations and UROXATRAL® was measured in 900 mL pH 4.5 Acetate buffer using a USP 2 apparatus with a 100 rpm paddle speed at 37 C.

TABLE 4
Comparative Dissolution Profile for Alfuzosin HCl ER
tablets of Example 1 and UROXATRAL, 10 mg in
pH 4.5 Acetate buffer using paddle at 100 rpm 900 mL

| | UROX- | | | | | | | | | UROX- |
|---|---|---|---|---|---|---|---|---|---|---|
| Time | ATROL | | | | | | | | | ATROL |
| (hr) | #25502 | I | II | III | V | VI | IV | VII | VIII | #25518 |
| 0 | 0 | 0 | 0 | 0 | 0 | 0 | 0 | 0 | 0 | 0 |
| 1 | 16 | 15 | 20 | 17 | 16 | 15 | 13 | 13 | 17 | 16 |
| 2 | 23 | 20 | 27 | 27 | 25 | 27 | 24 | 18 | 23 | 21 |
| 3 | 28 | 25 | 35 | 34 | 33 | 33 | 29 | 26 | 31 | 27 |
| 4 | 33 | 30 | 41 | 40 | 39 | 40 | 35 | 32 | 37 | 33 |
| 6 | 42 | 39 | 52 | 51 | 50 | 51 | 46 | 41 | 47 | 41 |
| 8 | 52 | 47 | 60 | 61 | 60 | 59 | 56 | 50 | 56 | 49 |
| 10 | 62 | 53 | 67 | 69 | 68 | 69 | 65 | 57 | 62 | 54 |
| 12 | 72 | 59 | 73 | 75 | 76 | 75 | 72 | 64 | 70 | 65 |
| 16 | 91 | 70 | 83 | 87 | 88 | 85 | 82 | 76 | 81 | 81 |
| 23 | 108 | 83 | 94 | 99 | 102 | 98 | 99 | 92 | 95 | 100 |

Dissolution of the Example 1 alfuzosin formulations and UROXATRAL® was also measured in 900 mL of pH 6.8 phosphate buffer in a USP 2 apparatus using Sandwich sinkers with a 100 rpm paddle speed at 37 C, as summarized below in Table 5.

TABLE 5
Dissolution Profiles of Formulations III-VIII and
UROXATRAL in pH 6.8 phosphate buffer

| Time | UROXATRAL | | | | | UROXATRAL | | |
|---|---|---|---|---|---|---|---|---|
| (hr) | #25502 | III | V | VI | IV | #25518 | VII | VIII |
| 0 | 0 | 0 | 0 | 0 | 0 | 0 | 0 | 0 |
| 1 | 15 | 14 | 14 | 15 | 12 | 13 | 10 | 12 |
| 2 | 20 | 20 | 21 | 23 | 19 | 19 | 15 | 19 |
| 3 | 25 | 25 | 27 | 29 | 24 | 23 | 20 | 25 |
| 4 | 29 | 30 | 32 | 34 | 28 | 27 | 24 | 30 |
| 6 | 37 | 38 | 41 | 43 | 37 | 33 | 32 | 38 |
| 8 | 44 | 46 | 49 | 50 | 44 | 39 | 38 | 46 |
| 10 | 52 | 52 | 55 | 58 | 51 | 44 | 44 | 52 |
| 12 | 57 | 57 | 62 | 64 | 58 | 50 | 50 | 58 |
| 16 | 72 | 67 | 72 | 72 | 65 | 62 | 64 | 74 |
| 23 | 89 | 81 | 86 | 87 | 84 | 80 | 74 | 81 |

### Example 3

### Dissolution of Example 1 Alfuzosin Formulations I-VI and UROXATRAL® Using USP 3 Apparatus

Dissolution of several of the Example 1 alfuzosin formulations I-VI and UROXATRAL® was measured in USP 3 apparatus (Bio Disc) at 12 dpm in 250 mL of 0.01N HCl (0-1 hr), Acetate Buffer pH 4.5 (2-3 hr) and pH 6.8 phosphate Buffer (9-23 hr) at 37 C.

TABLE 6
Dissolution Profiles for Formulations I-VI and
UROXATRAL using USP apparatus 3 at 12 dpm

| Time | UROXATRAL | | | | | | |
|---|---|---|---|---|---|---|---|
| (hr) | Lot # 25502 | I | II | III | IV | V | VI |
| 0 | 0 | 0 | 0 | 0 | 0 | 0 | 0 |
| 1 | 15 | 14 | 19 | 15 | 13 | 17 | 18 |
| 2 | 24 | 22 | 31 | 25 | 21 | 26 | 28 |
| 3 | 32 | 29 | 40 | 32 | 28 | 35 | 37 |
| 9 | 57 | 49 | 65 | 61 | 54 | 64 | 65 |
| 16 | 81 | 69 | 84 | 83 | 78 | 85 | 87 |
| 23 | 98 | 88 | 98 | 98 | 97 | 100 | 101 |

Additionally, dissolution of the Example 1 alfuzosin formulations and UROXATRAL® was measured in the USP 3 apparatus (Bio Disc) at 20 dpm in 250 mL of 0.01N HCl (0-1 hr), Acetate Buffer pH 4.5 (2-3 hr) and pH 6.8 phosphate Buffer (9-23 hr) at 37 C.

TABLE 7
Dissolution Profiles for Formulations I-VIII and
UROXATRAL using USP apparatus 3 at 20 dpm

| Time | Uroxatral | Formulation | Formulation | Formulation |
|---|---|---|---|---|
| (hrs) | # 25518 | IV | V | VI |
| 0 | 0 | 0 | 0 | 0 |
| 1 | 15 | 15 | 17 | 17 |
| 2 | 24 | 25 | 28 | 28 |
| 3 | 31 | 33 | 36 | 37 |
| 9 | 54 | 61 | 64 | 67 |
| 16 | 73 | 87 | 87 | 90 |
| 23 | 91 | 105 | 104 | 104 |

| | | | | | Formu- |
|---|---|---|---|---|---|
| Time | Formulation | Formulation | Formulation | Formulation | lation |
| (hrs) | III | I | II | VII | VIII |
| 0 | 0 | 0 | 0 | 0 | 0 |
| 1 | 17 | 14 | 20 | 13 | 16 |
| 2 | 27 | 22 | 33 | 22 | 26 |
| 3 | 35 | 29 | 43 | 29 | 34 |
| 9 | 65 | 52 | 72 | 55 | 62 |
| 16 | 88 | 71 | 93 | 76 | 83 |
| 23 | 103 | 88 | 107 | 92 | 98 |

### Example 4

### In Vivo Pharmacokinetic Parameters Under Fasted Administration Conditions for the Alfuzosin HCl Formulations I and II of Example 1 and UROXATRAL®

The purpose of this pilot study was to evaluate the relative bioavailability of the Example 1 formulations of alfuzosin HCl extended release 10 mg tablets with a marketed reference formulation UROXATRAL® 10 mg (alfuzosin hydrochloride extended release) tablets (Sanofi-Synthelabo), under fasted conditions in healthy male adult subjects.

A randomized, single-dose, three-treatment, three-way, crossover pilot study was conducted with up to 12 healthy, male adult subjects to compare two test alfuzosin HCl 10 mg extended release tablet formulations with that of an already marketed reference formulation, UROXATRAL® 10 mg (alfuzosin hydrochloride extended release) tablets (Sanofi-Synthelabo), under fasted conditions.

Test A: Formulation I Alfuzosin HCl Extended Release Tablets 10 mg

Test B: Formulation II Alfuzosin HCl Extended Release Tablets 10 mg

Reference C: UROXATRAL® 10 mg (alfuzosin HCl extended release) Tablets

In each period, one 10 mg tablet was administered to the subjects following an overnight fast of at least 10 hours. Each dose was accompanied by 240 mL (8 fl. ozs) of room temperature tap water. Subjects were instructed to swallow the tablet whole without chewing or biting. Subjects received the test products in two of the study periods and the reference product in the other period. The order of treatment administration followed a dosing randomization schedule. Each dose was separated by at least a 14-day interval.

In each study period, blood samples were obtained at hour 0 (pre-dose), and following the dose at hours 1, 2, 3, 3.5, 4, 4.5, 5, 5.5, 6, 6.5, 7, 7.5, 8, 8.5, 9, 10, 12, 18, 24, 36*, 48* and 72* hours post-dose, respectively (*samples requiring return visits from subjects). Standardized meals were served to all subjects at meals at approximately 4, 9 and 13 hours after dosing. Subjects consumed only the food and beverages provided. No caffeine, alcohol or grapefruit products were permitted during confinement. When meals and sample collections coincided, samples were collected before meals were served.

The samples were analyzed for alfuzosin in plasma by a fully validated analytical procedure. Statistical analysis was performed to evaluate the relative bioavailability of the test formulations to that of the reference product.

Plasma alfuzosin data were analyzed according to the FDA January 2001 guidance entitled Statistical Approaches to Establishing Bioequivalence using SAS® software (Release 9.13 or later). Area under the plasma concentration-time curve from time zero to time t of the last measurable concentration (AUC₀₋ₜ) was calculated by the linear trapezoidal method. Area under the plasma concentration-time curve from time zero to time infinity (AUC_{0-∞}) was calculated from the following: AUC_{0-∞}=AUC₀₋ₜ+Cₜ/Kₑₗ. The term Cₜ indicates the last measurable concentration and the term Kₑₗ indicates the terminal elimination rate, estimated from regression of the natural logarithms of the concentrations on the sampling times in the terminal elimination phase. Peak concentration (C_{MAX}) was obtained directly from the plasma concentration data. Time-to-peak concentration (T_{MAX}) was estimated as the first sampling time at which that peak concentration was reached.

Analyses of Variance were performed using the General Linear Model (GLM) procedure of SAS with hypothesis testing for treatment effects at α=0.05. The statistical model contained main effects of sequence, subject within sequence, treatment and period. Confidence intervals (90%) for the comparison of test and reference area and peak results were constructed to test two one-sided hypotheses at the α=0.05 level of significance. The confidence intervals were presented for the ratio of the test-to-reference treatment means, and for the geometric mean ratios (obtained from logarithmic transformation of the data).

Ratios of AUC, AUC_{0-∞}, C_{MAX}, ELIMHALF (drug elimination half life, sometimes expressed as T_{1/2}), KE (Drug elimination rate constant), and logarithmic transformed AUC₀₋ₜ (ng·h/mL), AUC_{0-∞}, (ng·h/mL), and C_{MAX} (ng/mL) comparing Example 1 formulations I or II to UROXATRAL® under fasting conditions, as well as the 90% upper and lower confidence limits on the ratios, are shown in Table 8 below.

TABLE 8

| | Formulation I | | | Formulation II | | |
|---|---|---|---|---|---|---|
| | (Test A)/UROXATRAL | | | (Test B)/UROXATRAL | | |
| PARAMETER | RATIO | CI Low | CI High | RATIO | CI Low | CI High |
| AUC | 0.7645 | 0.582 | 0.9471 | 0.9352 | 0.6673 | 1.2032 |
| AUCINF | 0.7025 | 0.5583 | 0.8468 | 0.891 | 0.6197 | 1.1623 |
| CMAX | 1.0056 | 0.806 | 1.2053 | 1.1854 | 0.9874 | 1.3835 |
| ELIMHALF | 1.0574 | 0.8425 | 1.2724 | 0.9572 | 0.6982 | 1.2161 |
| KE | 0.9159 | 0.7593 | 1.0724 | 1.0168 | 0.7613 | 1.2723 |
| LNAUC | 0.7842 | 0.6267 | 0.9811 | 0.9587 | 0.7187 | 1.2787 |
| LNAUCINF | 0.7088 | 0.6261 | 0.8025 | 0.8885 | 0.6694 | 1.1792 |
| LNCMAX | 1.0234 | 0.8266 | 1.267 | 1.2234 | 1.0038 | 1.491 |

Median Tₘₐₓ for Example 1 formulations I and II and for UROXATRAL determined in the fasting study were 5, 4.5, and 5.5 hr, respectively.

### Example 5

### In Vivo Pharmacokinetic Parameters Under Non-Fasted Administration Conditions for the Alfuzosin HCl Formulations I and II of Example 1 and UROXATRAL®

The purpose of this pilot study was to evaluate the relative bioavailability of the Example 1 formulations of alfuzosin HCl extended release 10 mg tablets with a marketed reference formulation UROXATRAL® 10 mg (alfuzosin hydrochloride extended release) tablets (Sanofi-Synthelabo), under non-fasted conditions in healthy male adult subjects. The study design and data collection and analysis was identical to that described above for the fasted study except that subjects consumed a standardized high fat breakfast prior to administration of the dose. The standard high fat breakfast was consumed starting approximately 30 minutes prior to dosing and consisted of: 2 eggs fried in butter, 2 strips of bacon, 4 oz hash brown potatoes, 2 slices of toast with butter, and 8 oz of whole milk. This meal contained approximately 150 protein calories, 250 carbohydrate calories and 500 fat calories.

Ratios of AUC, AUC_{0-∞}, Cₘₐₓ, ELIMHALF, KE, and logarithmic transformed AUC₀₋ₜ (ng·h/mL), AUC_{0-∞} (ng·h/mL), and Cₘₐₓ (ng/mL) comparing Example 1 formulations I or II to UROXATRAL® under non-fasting conditions, as well as the 90% upper and lower confidence limits on the ratios, are shown in Table 9 below.

TABLE 9

| | Formulation | | | Formulation | | |
|---|---|---|---|---|---|---|
| | I/UROXATRAL | | | II/UROXATRAL | | |
| PARAMETER | RATIO | CI Low | CI High | RATIO | CI Low | CI High |
| AUC | 0.9761 | 0.7728 | 1.1794 | 1.0707 | 0.8661 | 1.2753 |
| AUCINF | 0.9963 | 0.8001 | 1.1925 | 0.9971 | 0.817 | 1.1772 |
| CMAX | 0.9692 | 0.6474 | 1.2911 | 1.1112 | 0.9094 | 1.313 |
| ELIMHALF | 1.0841 | 0.8835 | 1.2848 | 0.9365 | 0.7176 | 1.1554 |
| KE | 0.8986 | 0.7265 | 1.0707 | 1.019 | 0.8267 | 1.2114 |
| LNAUC | 1.0728 | 0.8853 | 1.3001 | 1.1793 | 0.9677 | 1.4371 |
| LNAUCINF | 1.0795 | 0.888 | 1.3122 | 1.0872 | 0.8961 | 1.3191 |
| LNCMAX | 1.0754 | 0.7897 | 1.4645 | 1.2376 | 1.047 | 1.4629 |

Median Tₘₐₓ for Example 1 formulations I and II and for UROXATRAL determined in the non-fasting study were 6.25, 5.75, and 6.25 hr, respectively.

Comparison of the pharmacokinetic data collected in Examples 4 and 5 show that the commercially available product has a large food effect (i.e. the pharmacokinetic parameters increased significantly under non-fasting (fed) conditions vs fasting conditions). For UROXATRAL, Cₘₐₓ increased under non-fasting conditions ˜111% (9.4±1.0 (s.d.) ng/mL fasting vs. 19.1±3.4 (s.d.) ng/mL fed), while AUC increased under non-fasting conditions ˜36% (233.9±19.2 (s.d.) ng-h/mL fasting vs. 315.4±33.8 (s.d.) ng-h/mL fed). In contrast, Formulation I increased under non-fasting conditions: Cₘₐₓ˜100%, AUC˜91%; and Formulation II increased under non-fasting conditions: Cₘₐₓ˜91%, AUC˜52%.

### Example 6

### In Vivo Pharmacokinetic Parameters Under Fasted Administration Conditions for the Alfuzosin HCL Formulations IV and Viii of Example 1 and Uroxatral®

The purpose of this biostudy is to evaluate the relative bioavailability of the Example 1 formulations IV and VIII of alfuzosin HCl extended release 10 mg tablets with a marketed reference formulation UROXATRAL® 10 mg (alfuzosin hydrochloride extended release) tablets (Sanofi-Synthelabo), under fasted conditions in healthy male adult subjects.

A randomized, single-dose, two-way, crossover biostudy was conducted with 40 healthy, male adult subjects to compare two test alfuzosin HCl 10 mg extended release tablet formulations with that of an already marketed reference formulation, UROXATRAL® 10 mg (alfuzosin hydrochloride extended release) tablets (Sanofi-Synthelabo), under fasting conditions.

Test A: Formulation IV Alfuzosin HCl Extended Release Tablets 10 mg

Test B: Formulation VIII Alfuzosin HCl Extended Release Tablets 10 mg

Reference C: UROXATRAL® 10 mg (alfuzosin HCl extended release) Tablets

In each period, one 10 mg tablet was administered to the subjects following an overnight fast of at least 10 hours. Each dose was accompanied by 240 mL (8 fl. ozs) of room temperature tap water. Subjects were instructed to swallow the tablet whole without chewing or biting. Subjects received the test products in two of the study periods and the reference product in the other period. The order of treatment administration followed a dosing randomization schedule. Each dose was separated by at least a 14-day interval.

In each study period, blood samples were obtained at hour 0 (pre-dose), and following the dose at hours 1, 2, 3, 3.5, 4, 4.5, 5, 5.5, 6, 6.5, 7, 7.5, 8, 9, 10, 12, 16, 20, 24, 36 and 48 hours post-dose, respectively. Standardized meals were served to all subjects at approximately 4.25, 10.5 and 14.5 hours after dosing. Subjects consumed only the food and beverages provided. No caffeine, alcohol or grapefruit products were permitted during confinement. When meals and sample collections coincided, samples were collected before meals were served.

The plasma samples were analyzed for alfuzosin in plasma by a fully validated analytical procedure. The same statistical analysis as described in Example 4 was performed to evaluate the relative bioavailability of the test formulations to that of the reference product.

Ratios of AUC, AUC_{0-∞}, T_{MAX}, C_{MAX}, ELIMHALF (drug elimination half life, sometimes expressed as T_{1/2}), KE (Drug elimination rate constant), and logarithmic transformed AUC₀₋ₜ (ng·h/mL), AUC_{0-∞}, (ng·h/mL), and C_{MAX} (ng/mL) comparing Example 1 formulations IV or VIII to UROXATRAL® under fasting conditions, as well as the 90% upper and lower confidence limits on the ratios, are shown in Table 10 below.

TABLE 10
Summary of Statistical Analysis - fasted (N = 39)

| | Formulation | | | Formulation | | |
|---|---|---|---|---|---|---|
| | IV/UROXATRAL | | | VIII/UROXATRAL | | |
| PARAMETER | RATIO | CI Low | CI High | RATIO | CI Low | CI High |
| AUC | 0.8653 | 0.7589 | 0.9717 | 0.9816 | 0.8917 | 1.0716 |
| AUCINF | 0.9047 | 0.7945 | 1.015 | 0.9750 | 0.8887 | 1.0613 |
| TMAX | 0.6785 | 0.4209 | 0.9361 | 1.0083 | 0.8809 | 1.1357 |
| CMAX | 1.0920 | 0.9642 | 1.2198 | 1.3084 | 1.1892 | 1.4275 |
| ELIMHALF | 1.0473 | 0.8609 | 1.2336 | 1.0335 | 0.8229 | 1.2441 |
| KE | 1.0296 | 0.8339 | 1.2253 | 0.9461 | 0.7734 | 1.1189 |
| LNAUC | 0.8378 | 0.7441 | 0.9433 | 1.0005 | 0.9114 | 1.0984 |
| LNAUCINF | 0.8888 | 0.7926 | 0.9968 | 1.0014 | 0.915 | 1.096 |
| LNCMAX | 1.0725 | 0.9513 | 1.2092 | 1.2994 | 1.1854 | 1.4245 |

Non-Transformed Data for Formulation VIII

Least Squares Mean

| PK Variable | Test B | Reference |
|---|---|---|
| Cₘₐₓ | 10.29 | 7.87 |
| AUC₀₋ₜ | 128.54 | 130.94 |
| AUC_{0-∞} | 147.58 | 151.37 |
| Tₘₐₓ | 4.65 | 4.61 |
| kₑ | 0.0739 | 0.0781 |
| t_{1/2} | 12.30 | 11.90 |

Ln-Transformed Data for Formulation VIII

Geometric Mean

| PK Variable | Test B | Reference |
|---|---|---|
| Cₘₐₓ | 9.49 | 7.30 |
| AUC₀₋ₜ | 115.07 | 115.01 |
| AUC_{0-∞} | 135.00 | 134.81 |

Geometric means are based on least squares means of ln-transformed values.

### Example 7

### In Vivo Pharmacokinetic Parameters Under Non-Fasted Administration Conditions for the Alfuzosin HCl Formulations IV and VIII of Example 1 and UROXATRAL®

The purpose of this biostudy is to evaluate the relative bioavailability of the Example 1 formulations IV and VIII of alfuzosin HCl extended release 10 mg tablets with a marketed reference formulation UROXATRAL® 10 mg (alfuzosin hydrochloride extended release) tablets (Sanofi-Synthelabo), under non-fasted conditions in healthy male adult subjects.

The study design and data collection and analysis was identical to that described above in Example 6 for the fasted study except that subjects consumed a standardized high fat breakfast prior to administration of the dose. Fifty-two healthy male adults participated in this non-fasted study. The standard high fat breakfast was consumed starting approximately 30 minutes prior to dosing and consisted of: 2 eggs fried in butter, 2 strips of bacon, 4 oz hash brown potatoes, 2 slices of toast with butter, and 8 oz of whole milk. This meal contained approximately 150 protein calories, 250 carbohydrate calories and 500 fat calories.

Ratios of AUC, AUC_{0-∞}, T_{MAX}, Cₘₐₓ, ELIMHALF, KE, and logarithmic transformed AUC₀₋ₜ (ng·h/mL), AUC_{0-∞} (ng·h/mL), and Cₘₐₓ (ng/mL) comparing Example 1 formulations IV or VIII to UROXATRAL® under non-fasting conditions, as well as the 90% upper and lower confidence limits on the ratios, are shown in Table 11 below.

TABLE 11
Summary of Statistical Analysis - non-fasted (N = 51)

| | Formulation | | | Formulation | | |
|---|---|---|---|---|---|---|
| | IV/UROXATRAL | | | VIII/UROXATRAL | | |
| PARAMETER | RATIO | CI Low | CI High | RATIO | CI Low | CI High |
| AUC | 1.0990 | 1.0362 | 1.1618 | 0.9806 | 0.9091 | 1.0521 |
| AUCINF | 1.0859 | 1.0258 | 1.1461 | 0.9794 | 0.9132 | 1.0457 |
| TMAX | 0.8935 | 0.7228 | 1.0642 | 0.8396 | 0.7147 | 0.9645 |
| CMAX | 1.1373 | 1.0597 | 1.2148 | 1.0866 | 0.9992 | 1.174 |
| ELIMHALF | 0.9609 | 0.8745 | 1.0474 | 0.8737 | 0.804 | 0.9434 |
| KE | 1.0561 | 0.9773 | 1.135 | 1.1288 | 1.0564 | 1.2011 |
| LNAUC | 1.1123 | 1.0477 | 1.1808 | 0.9861 | 0.9181 | 1.0592 |
| LNAUCINF | 1.0943 | 1.0331 | 1.1592 | 0.9899 | 0.9289 | 1.0549 |
| LNCMAX | 1.1678 | 1.0893 | 1.252 | 1.1246 | 1.036 | 1.2208 |

Non-Transformed Data for Formulation VIII

Least Squares Mean

| PK Variable | Test | Reference |
|---|---|---|
| Cₘₐₓ | 16.57 | 15.25 |
| AUC₀₋ₜ | 208.71 | 212.83 |
| AUC_{0-∞} | 221.94 | 226.60 |
| Tₘₐₓ | 5.77 | 6.87 |
| Kₑ | 0.0847 | 0.0751 |
| t_{1/2} | 8.90 | 10.18 |

Ln-Transformed Data for Formulation VIII

Geometric Mean

| PK Variable | Test | Reference |
|---|---|---|
| Cₘₐₓ | 15.94 | 14.17 |
| AUC₀₋ₜ | 195.75 | 198.50 |
| AUC_{0-∞} | 209.83 | 211.97 |

Geometric means are based on least squares means of ln-transformed values.

### Example 8

### Alfuzosin Extended Release Co-Compressed Tablet (Tablet-in-Tablet) Formulation

The composition of Formulation VIII was also used to prepare a co-compressed tablet (tablet-in-tablet) extended release dosage form, as an alternative to the Formulation VIII compressed tablet investigated above. The alfuzosin extended release co-compressed tablet (tablet-in-tablet) formulation of this example comprises alfuzosin HCl as follows:

TABLE 12
Extended release co-compressed tablet (tablet-in-tablet)
formulation of alfuzosin HCl

| | | |
|---|---|---|
| Ingredient | Mg/Tablet | Wt % |

Core

| | | |
|---|---|---|
| Alfuzosin Hydrochloride | 2 | 0.57 |
| Avicel PH 101 | 54 | 15.43 |

Outer Coat

| | | |
|---|---|---|
| Alfuzosin Hydrochloride | 8 | 2.28 |
| HPMC K4MCR (Dow) | 175 | 50 |
| Ethyl cellulose T10 | 70 | 20 |
| HPMC K100LV | 35 | 10 |
| Magnesium stearate | 4 | 1.14 |
| Colloidal silicon dioxide, NF | 2 | 0.57 |
| Total | 350 | 100 |

The co-compressed tablet (tablet-in-tablet) formulation was prepared in the following manner. Alfuzosin Hydrochloride and Avicel PH 101 were sized with a #20 mesh screen and mixed to form a core blend. The core blend was compressed into tablet cores on a Carver press using 6/32 inch round SC tooling.

Alfuzosin Hydrochloride, HPMC K4MCR, Ethyl Cellulose T10, and HPMC K100LV were sized with a #20 mesh screen and mixed for 2 minutes to form a first blend. Magnesium stearate and colloidal silicon dioxide were sized with a #30 mesh screen and added to the first blend and mixed for 1 minute at low speed to form an outer coat blend. Then, the following materials were added to a die cavity of a Carver press in the following sequence −about 50 wt % of the outer coat blend (about 147 mg), the tablet core, and the rest of the outer coat blend (about 50 wt % of the outer coat mixture or about 147 mg). Finally, the content in the die was compressed on the Carver press using 12/32 inch FFBE tooling to form a co-compressed tablet (tablet-in-tablet) composition. The co-compressed tablet (tablet-in-tablet) composition has a hardness of about 20 kp.

### Example 9

### Dissolution of Example 8 Alfuzosin Co-Compressed Tablet (Tablet-in-Tablet) Formulation Using USP Type II Apparatus

The dissolution profile of the Example 8 alfuzosin co-compressed tablet (tablet-in-tablet) formulation was compared to the dissolution profiles of UROXATRAL® and the Formulation VIII compressed tablet of Example 1 in 900 mL pH 4.5 acetate buffer using a USP 2 apparatus with a 100 rpm paddle speed at 37 C.

TABLE 13
Dissolution Profiles for Example 8 co-compressed table
(tablet-in-tablet) formulation, Formulation VIII, and
UROXATRAL in pH 4.5 acetate buffer

| Time | Uroxatral tablet | Example 8 | Ex. 1, Formulaton VIII |
|---|---|---|---|
| (hr) | 10 mg | (Co-compressed Tablet) | (Single tablet) |
| 0 | 0 | 0 | 0 |
| 1 | 17 | 15 | 18 |
| 2 | 23 | 22 | 24 |
| 3 | 28 | 28 | 31 |
| 4 | 33 | 34 | 37 |
| 6 | 42 | 43 | 48 |
| 8 | 50 | 52 | 57 |
| 10 | 57 | 59 | 65 |
| 12 | 64 | 65 | 72 |
| 16 | 79 | 78 | 85 |

The Example 8 alfuzosin co-compressed tablet (tablet-in-tablet) formulation has a dissolution profile that is substantially identical to a dissolution profile of UROXATRAL®.

### Example 10

### Alfuzosin Coated Formulations

TABLE 14
Alfuzosin Core composition

| NB1460:01 | mg/tab | % |
|---|---|---|
| Alfuzosin Hydrochloride | 10 | 5 |
| HPMC K4MCR | 80 | 40 |
| HPMC K100LV | 30 | 15 |
| Ethylcellulose T10 | 50 | 25 |
| Avicel PH101 | 26 | |
| Colloidal silicon dioxide, NF | 2 | |
| Magnesium Stearate | 2 | |
| Total tablet weight | 200 | |

The core tablet is formed from the ingredients listed in Table 14 as described above in Example 1. The compressed tablets are coated with a coating prepared from a suspension of Surelease®, Opadry® Clear and water to form extended release alfuzosin tablets. Two different coated formulations are shown in Table 15 below.

TABLE 15
Alfuzosin coated formulations

| | NB1460:06 | NB1460:08 |
|---|---|---|
| | IX | X |
| | mg/tab | mg/tab |
| Tablet core | | |
| Alfuzosin Hydrochloride | 10 | 10 |
| HPMC K4MCR | 80 | 80 |
| HPMC K100LV | 30 | 30 |
| Ethylcellulose T10 | 50 | 50 |
| Avicel PH101 | 26 | 26 |
| Colloidal silicon dioxide, NF | 2 | 2 |
| Magnesium Stearate | 2 | 2 |
| Coating composition | | |
| Surelease | 8 | 4.8 |
| Opadry Clear | 8 | 11.2 |
| Total tablet weight | 216 | 216 |

The coated formulations of Table 15 can be analyzed for in vitro dissolution using the test method protocol according to USP 26, <711> test apparatus 2 (paddle) with a 100 rotations per minute (rpm) paddle speed at 37° C.±0.5° C. using sequential dissolution media: 500 ml 0.1N HCL (0-1 hr), 250 ml 0.350M Sodium Acetate (2-3 hr), and 250 ml 0.125M Tribasic sodium phosphate (4-23 hr). Results are shown in Table 16 below.

TABLE 16
Sequential dissolution: 500 ml 0.1N HCL + 250 ml 0.350M Sodium
Acetate + 250 ml of 0.125M Tribasic sodium phosphate; App# 2
(paddle); 100 rpm, Sandwitch sinker.

| | | 0.1N | pH 4.5 | | | | | | | | |
|---|---|---|---|---|---|---|---|---|---|---|---|
| | | HCL | buffer | | PH 6.8 buffer | | | | | | |
| | Time (hrs) | | | | | | | | | | |
| | 0 | 1 | 2 | 3 | 4 | 6 | 8 | 10 | 12 | 16 | 23 |
| UROXATRAL | 0 | 14 | 20 | 24 | 28 | 37 | 45 | 48 | 51 | 59 | 72 |
| Tablet Core | 0 | 19 | 30 | 38 | 44 | 60 | 68 | 71 | 76 | 82 | 88 |
| (NB1460:01) | | | | | | | | | | | |
| IX | 0 | 11 | 20 | 28 | 35 | 47 | 55 | 62 | 68 | 77 | 95 |
| (NB1460:06) | | | | | | | | | | | |
| X (NB1460:08) | 0 | 16 | 27 | 34 | 41 | 54 | 60 | 67 | 73 | 82 | 95 |

Additional coated formulations were prepared using a different core tablet. In these coated formulations, the composition of the core tablet was as shown below in Table 17.

TABLE 17
Alfuzosin Core composition

| NB1467:15 BB IM0630417 | mg/tab | % |
|---|---|---|
| Alfuzosin Hydrochloride | 10 | 5 |
| HPMC K4MCR | 70 | 35 |
| HPMC K100LV | 20 | 10 |
| Ethylcellulose T10 | 70 | 35 |
| Avicel PH101 | 26 | 13 |
| Colloidal silicon dioxide, NF | 2 | |
| Magnesium Stearate | 2 | |
| Total tablet weight | 200 | |

The core tablet were formed from the ingredients listed in Table 17 as described above in Example 1. The compressed tablets were then coated with a coating prepared from a suspension of Surelease®, Opadry® Clear and water until a desired weight gain was observed for the tablet to obtain extended release tablets. The coating mixture consisted of 3.6 kg SURERELEASE (E-7-1910), 0.9 kg OPADRY CLEAR mixed in 13.5 kg purified water. SURELEASE (E-7-1910) is supplied as a 25% w/w aqueous dispersion of Ethylcellulose. Tablets were sprayed with the coating mixture to obtain the following weight gains per tablet: 2%, 6%, 14%, and 16%. Relative bioavailability studies on the tablets coated to obtain a 6% weight gain or a 14% weight gain are described below.

### Example 11

### Relative Bioavailability Under Fasting and Fed Conditions of the Table 17 10 mg Alfuzosin HCl Core Tablets Coated to Obtain a 6% Weight Gain and of 10 mg UROXATRAL Tablets Under Fed Conditions

An open-label, randomized, single-dose, three-period, three-treatment study was performed with 54 healthy male adult subjects to determine relative bioavailability of the Table 17 Alfuzosin HCl core tablets coated to obtain a 6% weight gain (“6% weight gain coated Alfuzosin HCl tablets”) under fasting and fed conditions and of 10 mg UROXATRAL tablets under fed conditions. Each subject participated in three dosing periods separated by a washout period of fourteen days. The three dosing regimens will be one 10 mg 6% weight gain coated alfuzosin HCl tablet (test product A) administered with 240 mL room temperature water 30 minutes after initiation of a standardized, high-fat and high-calorie breakfast preceded by an overnight fast; one 10 mg 6% weight gain coated alfuzosin HCl tablet (test product B) administered with 240 mL room temperature water after an overnight fast of at least 10 hour; or one 10 mg reference UROXATRAL tablet (reference product C) administered with 240 mL room temperature water 30 minutes after initiation of a standardized, high-fat and high-calorie breakfast preceded by an overnight fast. Subjects will not be permitted to lie down for the first 4 hours following administration of the treatment to ensure proper stomach emptying.

Subjects will be confined in the early evening prior to and until at least 24 hours after each dose for each study period to control fluid and food intake. Subjects will return to the clinical site on a non-confined bases for continued pharmacokinetic blood sampling at 36, 38, and 72 hours post-dose in each period. No grapefruit or grapefruit-containing products, alcochol, or caffeine- or xanthine-containing products were allowed 48 hours before dosing on Day 1 and throughout the study. In each dosing period, blood samples will be drawn from each subject for drug content analysis within one hour prior to dosing (0 hour) and after dose administration at 0.5, 1, 1.5, 2, 2.5, 3, 3.5, 4, 4.5, 5, 5.5, 6, 7, 8, 12, 16, 20, 24, 36, 48, and 72 hours. Alfusozin plasma concentrations in the blood samples will be measured using a validated bioanalytical method.

The alfuzosin concentration-time data will be used to calculate the following pharmacokinetic parameters: AUC₀₋ₜ, AUC_{0-∞}, Cₘₐₓ, Tₘₐₓ, kₑₗ, t_{1/2}, Vₐᵣₑₐ/F, and CL/F. The pharmacokinetic parameters will be calculated using the software package WINNONLIN®, Model 200. Analyses for AUC₀₋ₜ, AUC_{0-∞}, and Cₘₐₓ will be performed on ln-transformed data. Analysis of variance (ANOVA) will be performed on the ln-transformed AUC₀₋ₜ, AUC_{0-∞}, and Cₘₐₓ.

The following results were obtained in a study performed in general accordance with the above protocol.

Summary of Statistical Analysis-DATA from RO8-373 study
Treatment A: Test (Fed) vs. Treatment C: Reference (Fed)
Ln-Transformed Data

| | | | | | | | 90% Confidence |
|---|---|---|---|---|---|---|---|
| | Least | | | | | | Interval |
| | Squares Mean | | Geometric Mean | | | Mean Square | (Lower Limit, |
| PK Variable | A | C | A | C | % Ratio | Error | Upper Limit) |
| Cₘₐₓ | 9.864 | 9.588 | 19225.53 | 14590.96 | 131.76 | 0.09234 | (119.35, 145.47) |
| AUC₀₋ₜ | 12.231 | 12.080 | 204972.82 | 176269.03 | 116.28 | 0.06376 | (107.11, 126.25) |
| AUC_{0-∞} | 12.286 | 12.156 | 216637.51 | 190188.84 | 113.91 | 0.05217 | (105.74, 122.7) |

Non-Transformed Data

| | | | | | 90% Confidence |
|---|---|---|---|---|---|
| | | | | | Interval |
| | Least Squares Mean | | | | (Lower Limit, |
| PK Variable | A | C | % Ratio | Mean Square Error | Upper Limit) |
| Cₘₐₓ | 20288.44 | 15827.16 | 128.19 | 19866980.07 | (119.02, 137.36) |
| AUC₀₋ₜ | 220369.06 | 192950.70 | 114.21 | 1890523645.80 | (106.87, 121.55) |
| AUC_{0-∞} | 231283.59 | 205825.50 | 112.37 | 1907599212.30 | (105.46, 119.28) |
| Tₘₐₓ | 5.36 | 6.73 | 79.64 | 4.2594 | (69.65, 89.63) |
| Kel | 0.1019 | 0.0922 | 110.42 | 0.00053 | (102.29, 118.56) |
| t_{1/2} | 7.31 | 8.39 | 87.19 | 6.5278 | (77.27, 97.11) |
| Vₐᵣₑₐ/F | 514090.84 | 677755.47 | 75.85 | 135512953544.0000 | (58.17, 93.54) |
| CL/F | 50270.47 | 57678.07 | 87.16 | 471890618.7600 | (74.89, 99.42) |
| Weight-Adjusted | 620.24 | 710.42 | 87.31 | 81554.5500 | (74.22, 100.39) |
| CL/F | | | | | |

Geometric means are based on least squares means of ln-transformed values.
P-value of Tmax calculated using Wilcoxon Signed Rank Test is 0.0029

Summary of Statistical Analysis
Treatment B: Test (Fasted) vs. Treatment A: Test (Fed) N = 52
Ln-Transformed Data

| | | | | | | | 90% Confidence |
|---|---|---|---|---|---|---|---|
| | Least | | | | | | Interval |
| | Squares Mean | | Geometric Mean | | | Mean Square | (Lower Limit, |
| PK Variable | B | A | B | A | % Ratio | Error | Upper Limit) |
| Cₘₐₓ | 9.124 | 9.864 | 9173.41 | 19225.53 | 47.71 | 0.09234 | (43.22, 52.68) |
| AUC₀₋ₜ | 11.730 | 12.231 | 124243.98 | 204972.82 | 60.61 | 0.06376 | (55.83, 65.81) |
| AUC_{0-∞} | 11.850 | 12.286 | 140049.01 | 216637.51 | 64.65 | 0.05217 | (60.01, 69.64) |

Non-Transformed Data

| | | | | | 90% Confidence |
|---|---|---|---|---|---|
| | | | | | Interval |
| | Least Squares Mean | | | | (Lower Limit, |
| PK Variable | B | A | % Ratio | Mean Square Error | Upper Limit) |
| Cₘₐₓ | 10034.45 | 20288.44 | 49.46 | 19866980.07 | (42.31, 56.61) |
| AUC₀₋ₜ | 135209.73 | 220369.06 | 61.36 | 1890523645.80 | (54.93, 67.78) |
| AUC_{0-∞} | 149555.33 | 231283.59 | 64.66 | 1907599212.30 | (58.51, 70.81) |
| Tₘₐₓ | 4.87 | 5.36 | 90.95 | 4.2594 | (78.41, 103.5) |
| Kel | 0.0801 | 0.1019 | 78.67 | 0.00053 | (71.31, 86.04) |
| t_{1/2} | 9.83 | 7.31 | 134.43 | 6.5278 | (123.05, 145.8) |
| Vₐᵣₑₐ/F | 1081554.26 | 514090.84 | 210.38 | 135512953544.0000 | (187.07, 233.7) |
| CL/F | 78158.36 | 50270.47 | 155.48 | 471890618.7600 | (141.41, 169.55) |
| Weight-Adjusted | 968.10 | 620.24 | 156.08 | 81554.5500 | (141.09, 171.08) |
| CL/F | | | | | |

Geometric means are based on least squares means of ln-transformed values.
P-value of Tmax calculated using Wilcoxon Signed Rank Test is 0.2319

TABLE 14.2.4
Individual Pharmacokinetic Parameters - Treatment A: Test (Fed)

| | AUC₀₋ₜ | AUC_{0-∞} | AUC₀₋ₜ/ | Cₘₐₓ | Tₘₐₓ | Kel | t_{1/2} | Vₐᵣₑₐ/F |
|---|---|---|---|---|---|---|---|---|
| Subject | (pg-hr/mL) | (pg-hr/mL) | AUC_{0-∞} | (pg/mL) | (hr) | (1/hr) | (hr) | (mL) |
| N | 52 | 52 | 52 | 52 | 52 | 52 | 52 | 52 |
| MEAN | 220707.66 | 231649.37 | 0.95 | 20311.96 | 5.36 | 0.10 | 7.31 | 511989.24 |
| STDEV | 79718.62 | 79578.88 | 0.03 | 6413.64 | 1.97 | 0.03 | 2.10 | 242836.18 |
| % CV | 36.12 | 34.35 | 3.44 | 31.58 | 36.80 | 26.13 | 28.73 | 47.43 |
| MEDIAN | 218304.25 | 229308.98 | 0.96 | 20055 | 4.5 | 0.0967 | 7.17 | 445410.805 |
| MIN | 59267.05 | 66131.95 | 0.86 | 6061 | 2.5 | 0.0494 | 3.92 | 215370.29 |
| MAX | 444428.60 | 450643.85 | 0.99 | 39950 | 10 | 0.1767 | 14.04 | 1509607.16 |

| | | Weight- | | | |
|---|---|---|---|---|---|
| | | adjusted | | | |
| | CL/F | CL/F | Ln-Transformed | | |
| Subject | (mL/hr) | (mL/hr/kg) | AUC₀₋ₜ | AUC_{0-∞} | Cₘₐₓ |
| N | 52 | 52 | 52 | 52 | 52 |
| MEAN | 50112.43 | 617.62 | 12.23 | 12.29 | 9.87 |
| STDEV | 23470.44 | 325.49 | 0.40 | 0.38 | 0.34 |
| % CV | 46.84 | 52.70 | 3.29 | 3.10 | 3.47 |
| MEDIAN | 43735.37 | 526.795 | 12.293633 | 12.342826 | 9.9062248 |
| MIN | 22181.6 | 246.46 | 10.989809 | 11.099407 | 8.7096301 |
| MAX | 154894.32 | 2139.42 | 13.004545 | 13.018433 | 10.595384 |

TABLE 14.2.5
Individual Pharmacokinetic Parameters - Treatment B: Test (Fasted)

| | | | | | | | | | | Weight- | | | |
|---|---|---|---|---|---|---|---|---|---|---|---|---|---|
| | | | | | | | | | | adjusted | | | |
| | AUC₀₋ₜ | AUC_{0-∞} | | | | | | | | CL/F | | | |
| | (pg-hr/ | (pg-hr/ | AUC₀₋ₜ/ | Cₘₐₓ | Tₘₐₓ | Kel | t_{1/2} | Vₐᵣₑₐ/F | CL/F | (mL/ | Ln-Transformed | | |
| Subject | mL) | mL) | AUC_{0-∞} | (pg/mL) | (hr) | (1/hr) | (hr) | (mL) | (mL/hr) | hr/kg) | AUC₀₋ₜ | AUC_{0-∞} | Cₘₐₓ |
| N | 52 | 51 | 51 | 52 | 52 | 51 | 51 | 52 | 52 | 52 | 52 | 51 | 52 |
| MEAN | 135399.75 | 150506.34 | 0.89 | 10072.00 | 4.87 | 0.08 | 9.81 | 1078172.61 | 78038.02 | 965.74 | 11.73 | 11.85 | 9.13 |
| STDEV | 51068.23 | 51362.91 | 0.08 | 4250.78 | 1.33 | 0.03 | 4.09 | 621518.16 | 41595.82 | 578.06 | 0.44 | 0.40 | 0.44 |
| % CV | 37.72 | 34.13 | 8.95 | 42.20 | 27.31 | 33.07 | 41.71 | 57.65 | 53.30 | 59.86 | 3.78 | 3.35 | 4.82 |
| ME- | 135436.75 | 150597.53 | 0.92 | 9294.5 | 4.5 | 0.0824 | 8.41 | 935287.755 | 67121.3 | 795.6 | 11.81584 | 11.922366 | 9.137148 |
| DIAN | | | | | | | | | | | | | |
| MIN | 30272.85 | 36154.42 | 0.63 | 2758 | 2 | 0.0279 | 4.42 | 417508.79 | 39751.26 | 427.43 | 10.31801 | 10.495554 | 7.922261 |
| MAX | 238590.00 | 251552.04 | 0.97 | 21390 | 9 | 0.1567 | 24.86 | 3241000.7 | 292659.09 | 4116.16 | 12.3825 | 12.435405 | 9.970679 |

TABLE 14.2.6
Individual Pharmacokinetic Parameters - Treatment C: Reference (Fed)

| | AUC₀₋ₜ | AUC_{0-∞} | AUC₀₋ₜ/ | Cₘₐₓ | Tₘₐₓ | Kel | t_{1/2} | Vₐᵣₑₐ/F |
|---|---|---|---|---|---|---|---|---|
| | (pg-hr/mL) | (pg-hr/mL) | AUC_{0-∞} | (pg/mL) | (hr) | (1/hr) | (hr) | (mL) |
| N | 52 | 52 | 52 | 52 | 52 | 52 | 52 | 52 |
| MEAN | 193346.19 | 206256.06 | 0.93 | 15871.62 | 6.72 | 0.09 | 8.38 | 675788.12 |
| STDEV | 81641.98 | 82053.15 | 0.06 | 6322.98 | 3.05 | 0.03 | 3.00 | 351438.74 |
| % CV | 42.23 | 39.78 | 6.74 | 39.84 | 45.44 | 31.66 | 35.74 | 52.00 |
| MEDIAN | 191134.60 | 200580.45 | 0.96 | 15470 | 5.5 | 0.08885 | 7.8 | 617895.37 |
| MIN | 52796.25 | 69265.41 | 0.72 | 5028 | 1.5 | 0.0429 | 4.2 | 171531.59 |
| MAX | 456650.00 | 468120.79 | 0.98 | 31650 | 12 | 0.165 | 16.16 | 2004341.91 |

| | | Weight- | | | |
|---|---|---|---|---|---|
| | | adjusted | | | |
| | CL/F | CL/F | Ln-Transformed | | |
| | (mL/hr) | (mL/hr/kg) | AUC₀₋ₜ | AUC_{0-∞} | Cₘₐₓ |
| N | 52 | 52 | 52 | 52 | 52 |
| MEAN | 57570.32 | 708.47 | 12.08 | 12.16 | 9.59 |
| STDEV | 26787.72 | 354.34 | 0.44 | 0.41 | 0.42 |
| % CV | 46.53 | 50.01 | 3.66 | 3.38 | 4.38 |
| MEDIAN | 49916.025 | 617.375 | 12.16073 | 12.208971 | 9.646617 |
| MIN | 21339.04 | 237.1 | 10.8742 | 11.145701 | 8.522778 |
| MAX | 53588.83 | 2121.39 | 13.03167 | 13.056482 | 10.36249 |

### Example 12

### Relative Bioavailability Under Fasting and Fed Conditions of the Table 17 10 mg Alfuzosin HCl Core Tablets Coated to Obtain a 14% Weight Gain and of 10 Mg UROXATRAL Tablets Under Fed Conditions

An open-label, randomized, single-dose, three-period, three-treatment study was performed with 54 healthy male adult subjects to determine relative bioavailability of the Table 17 Alfuzosin HCl core tablets coated to obtain a 14% weight gain (“14% weight gain coated Alfuzosin HCl tablets”) under fasting and fed conditions and of 10 mg UROXATRAL tablets under fed conditions. Each subject participated in three dosing periods separated by a washout period of fourteen days. The three dosing regimens will be one 10 mg 14% weight gain coated alfuzosin HCl tablet (test product A) administered with 240 mL room temperature water 30 minutes after initiation of a standardized, high-fat and high-calorie breakfast preceded by an overnight fast; one 10 mg 14% weight gain coated alfuzosin HCl tablet (test product B) administered with 240 mL room temperature water after an overnight fast of at least 10 hour; or one 10 mg reference UROXATRAL tablet (reference product C) administered with 240 mL room temperature water 30 minutes after initiation of a standardized, high-fat and high-calorie breakfast preceded by an overnight fast. Subjects will not be permitted to lie down for the first 4 hours following administration of the treatment to ensure proper stomach emptying.

Subjects will be confined in the early evening prior to and until at least 24 hours after each dose for each study period to control fluid and food intake. Subjects will return to the clinical site on a non-confined bases for continued pharmacokinetic blood sampling at 36, 38, and 72 hours post-dose in each period. No grapefruit or grapefruit-containing products, alcohol, or caffeine- or xanthine-containing products were allowed 48 hours before dosing on Day 1 and throughout the study. In each dosing period, blood samples will be drawn from each subject for drug content analysis within one hour prior to dosing (0 hour) and after dose administration at 0.5, 1, 1.5, 2, 2.5, 3, 3.5, 4, 4.5, 5, 5.5, 6, 7, 8, 12, 16, 20, 24, 36, 48, and 72 hours. Alfusozin plasma concentrations in the blood samples will be measured using a validated bioanalytical method.

The alfuzosin concentration-time data will be used to calculate the following pharmacokinetic parameters: AUC₀₋ₜ, AUC₀₋₂₈, Cₘₐₓ, Tₘₐₓ, Kₑₗ, t^{1/2}, Vₐᵣₑₐ/F, and CL/F. The pharmacokinetic parameters will be calculated using the software package WINNONLIN®, Model 200. Analyses for AUC₀₋ₜ, AUC_{0-∞}, and Cₘₐₓ will be performed on ln-transformed data. Analysis of variance (ANOVA) will be performed on the ln-transformed AUC₀₋ₜ, AUC_{0-∞}, and Cₘₐₓ.

The following results were obtained in a study performed in general accordance with the above protocol.

TABLE 14.2.13
Summary of Statistical Analysis-Data from R08-374
Treatment A: Test (Fed) vs. Treatment C: Reference (Fed) N = 53
Ln-Transformed Data

| | | | | | | | 90% Confidence |
|---|---|---|---|---|---|---|---|
| | Least | | | | | | Interval |
| | Squares Mean | | Geometric Mean | | | Mean Square | (Lower Limit, |
| PK Variable | A | C | A | C | % Ratio | Error | Upper Limit) |
| Cₘₐₓ | 9.628 | 9.582 | 15188.56 | 14494.68 | 104.79 | 0.12280 | (93.59, 117.32) |
| AUC₀₋ₜ | 12.040 | 12.054 | 169366.62 | 171775.74 | 98.60 | 0.07578 | (90.22, 107.75) |
| AUC_{0-∞} | 12.114 | 12.135 | 182396.61 | 186334.02 | 97.89 | 0.06225 | (90.32, 106.09) |

Non-Transformed Data

| | | | | | 90% Confidence |
|---|---|---|---|---|---|
| | | | | | Interval |
| | Least Squares Mean | | | | (Lower Limit, |
| PK Variable | A | C | % Ratio | Mean Square Error | Upper Limit) |
| Cₘₐₓ | 16998.85 | 16186.75 | 105.02 | 26486487.56 | (94.76, 115.27) |
| AUC₀₋ₜ | 187373.78 | 189090.40 | 99.09 | 1904413628.50 | (91.65, 106.53) |
| AUC_{0-∞} | 199567.68 | 201974.54 | 98.81 | 1907329048.90 | (91.83, 105.78) |
| Tₘₐₓ | 5.50 | 6.57 | 83.77 | 7.0985 | (70.69, 96.84) |
| Kel | 0.0972 | 0.1007 | 96.51 | 0.00067 | (88.2, 104.82) |
| t_{1/2} | 7.82 | 7.79 | 100.40 | 7.4218 | (89.12, 111.67) |
| Vₐᵣₑₐ/F | 678068.93 | 647609.83 | 104.70 | 185365943844.0000 | (83.26, 126.14) |
| CL/F | 60344.83 | 58499.92 | 103.15 | 563197709.5100 | (90.07, 116.24) |
| Weight-Adjusted | 727.58 | 717.54 | 101.40 | 75326.5807 | (89.07, 113.73) |
| CL/F | | | | | |

Geometric means are based on least squares means of ln-transformed values.
P-value of Tmax calculated using Wilcoxon Signed Rank Test is 0.0200.

TABLE 14.2.14
Summary of Statistical Analysis
Treatment B: Test (Fasted) vs. Treatment A: Test (Fed) N = 53
Ln-Transformed Data

| | | | | | | | 90% Confidence |
|---|---|---|---|---|---|---|---|
| | Least | | | | | | Interval |
| | Squares Mean | | Geometric Mean | | | Mean Square | (Lower Limit, |
| PK Variable | B | A | B | A | % Ratio | Error | Upper Limit) |
| Cₘₐₓ | 8.811 | 9.628 | 6707.11 | 15188.56 | 44.16 | 0.12280 | (39.44, 49.44) |
| AUC₀₋ₜ | 11.440 | 12.040 | 92920.69 | 169366.62 | 54.86 | 0.07578 | (50.2, 59.96) |
| AUC_{0-∞} | 11.620 | 12.114 | 111318.18 | 182396.61 | 61.03 | 0.06225 | (56.31, 66.15) |

Non-Transformed Data

| | | | | | 90% Confidence |
|---|---|---|---|---|---|
| | Least | | | | Interval |
| | Squares Mean | | | | (Lower Limit, |
| PK Variable | B | A | % Ratio | Mean Square Error | Upper Limit) |
| Cₘₐₓ | 7253.48 | 16998.85 | 42.67 | 26486487.56 | (32.91, 52.43) |
| AUC₀₋ₜ | 99991.99 | 187373.78 | 53.36 | 1904413628.50 | (45.86, 60.87) |
| AUC_{0-∞} | 114931.46 | 199567.68 | 57.59 | 1907329048.90 | (50.53, 64.65) |
| Tₘₐₓ | 5.57 | 5.50 | 101.25 | 7.0985 | (85.64, 116.86) |
| Kel | 0.0727 | 0.0972 | 74.86 | 0.00067 | (66.25, 83.47) |
| t_{1/2} | 10.89 | 7.82 | 139.22 | 7.4218 | (127.98, 150.45) |
| Vₐᵣₑₐ/F | 1491198.00 | 678068.93 | 219.92 | 185365943844.0000 | (199.44, 240.39) |
| CL/F | 92182.41 | 60344.83 | 152.76 | 563197709.5100 | (140.08, 165.44) |
| Weight-Adjusted | 1138.22 | 727.58 | 156.44 | 75326.5807 | (144.27, 168.6) |
| CL/F | | | | | |

Geometric means are based on least squares means of ln-transformed values.
P-value of Tmax calculated using Wilcoxon Signed Rank Test is 0.5828

TABLE 14.2.4
Individual Pharmacokinetic Parameters - Treatment A: Test (Fed)

| | AUC₀₋ₜ | AUC_{0-∞} | AUC₀₋ₜ/ | Cₘₐₓ | Tₘₐₓ | Kel | t_{1/2} | Vₐᵣₑₐ/F |
|---|---|---|---|---|---|---|---|---|
| | (pg-hr/mL) | (pg-hr/mL) | AUC_{0-∞} | (pg/mL) | (hr) | (1/hr) | (hr) | (mL) |
| N | 53 | 52 | 52 | 53 | 53 | 52 | 52 | 53 |
| MEAN | 187830.18 | 201250.56 | 0.93 | 17046.57 | 5.50 | 0.10 | 7.89 | 678254.52 |
| STDEV | 89443.05 | 89217.21 | 0.05 | 8113.61 | 2.52 | 0.03 | 2.61 | 367577.56 |
| % CV | 47.62 | 44.33 | 5.28 | 47.60 | 45.91 | 30.01 | 33.07 | 54.19 |
| MEDIAN | 162635.00 | 177342.52 | 0.94 | 16150 | 4.5 | 0.0969 | 7.155 | 536093.97 |
| MIN | 37019.05 | 41513.07 | 0.75 | 4503 | 1.5 | 0.0468 | 3.89 | 219053.55 |
| MAX | 475185.50 | 492176.84 | 0.98 | 39240 | 16 | 0.1782 | 14.81 | 1640927.07 |

| | | Weight- | | | |
|---|---|---|---|---|---|
| | | adjusted | | | |
| | CL/F | CL/F | Ln-Transformed | | |
| | (mL/hr) | (mL/hr/kg) | AUC₀₋ₜ | AUC_{0-∞} | Cₘₐₓ |
| N | 53 | 53 | 53 | 52 | 53 |
| MEAN | 60297.62 | 726.78 | 12.04 | 12.12 | 9.63 |
| STDEV | 32159.29 | 320.90 | 0.46 | 0.43 | 0.49 |
| % CV | 53.33 | 44.15 | 3.82 | 3.54 | 5.09 |
| MEDIAN | 56985.25 | 715.01 | 11.999264 | 12.085783 | 9.6896753 |
| MIN | 20317.9 | 258.54 | 10.519188 | 10.633764 | 8.4124991 |
| MAX | 240887.96 | 2189.89 | 13.071461 | 13.106593 | 10.577452 |

TABLE 14.2.5
Individual Pharmacokinetic Parameters - Treatment B: Test (Fasted)

| | AUC₀₋ₜ | AUC_{0-∞} | AUC₀₋ₜ/ | Cₘₐₓ | Tₘₐₓ | Kel | t_{1/2} | Vₐᵣₑₐ/F |
|---|---|---|---|---|---|---|---|---|
| | (pg-hr/mL) | (pg-hr/mL) | AUC_{0-∞} | (pg/mL) | (hr) | (1/hr) | (hr) | (mL) |
| N | 53 | 48 | 48 | 53 | 53 | 48 | 48 | 52 |
| MEAN | 100083.24 | 119555.02 | 0.85 | 7263.60 | 5.58 | 0.08 | 10.61 | 1485126.60 |
| STDEV | 39606.66 | 39417.02 | 0.11 | 3059.42 | 3.02 | 0.03 | 4.25 | 736351.31 |
| % CV | 39.57 | 32.97 | 13.03 | 42.12 | 54.11 | 40.85 | 40.05 | 49.58 |
| MEDIAN | 97270.35 | 118442.66 | 0.88 | 6766 | 4.5 | 0.0699 | 9.915 | 1391993.99 |
| MIN | 36126.35 | 54946.45 | 0.51 | 2943 | 3 | 0.027 | 4.42 | 416768.15 |
| MAX | 233854.50 | 249775.92 | 0.98 | 16460 | 24 | 0.1567 | 25.71 | 3760942.56 |

| | | Weight- | | | |
|---|---|---|---|---|---|
| | | adjusted | | | |
| | CL/F | CL/F | Ln-Transformed | | |
| | (mL/hr) | (mL/hr/kg) | AUC₀₋ₜ | AUC_{0-∞} | Cₘₐₓ |
| N | 52 | 52 | 53 | 47 | 53 |
| MEAN | 92127.00 | 1137.58 | 11.44 | 11.65 | 8.81 |
| STDEV | 31277.99 | 424.55 | 0.39 | 0.31 | 0.40 |
| % CV | 33.95 | 37.32 | 3.41 | 2.70 | 4.52 |
| MEDIAN | 84431.425 | 1054.29 | 11.48525 | 11.687483 | 8.819665 |
| MIN | 40035.88 | 479.47 | 10.49478 | 10.936938 | 7.987185 |
| MAX | 181995.37 | 2359.26 | 12.36245 | 12.428319 | 9.708688 |

TABLE 14.2.6
Individual Pharmacokinetic Parameters - Treatment C: Reference (Fed)

| | | | | | | | | | | Weight- | | | |
|---|---|---|---|---|---|---|---|---|---|---|---|---|---|
| | | | | | | | | | | adjusted | | | |
| | AUC₀₋ₜ | AUC_{0-∞} | | | | | | | | CL/F | | | |
| | (pg-hr/ | (pg-hr/ | AUC₀₋ₜ/ | Cₘₐₓ | Tₘₐₓ | Kel | t_{1/2} | Vₐᵣₑₐ/F | CL/F | (mL/ | Ln-Transformed | | |
| | mL) | mL) | AUC_{0-∞} | (pg/mL) | (hr) | (1/hr) | (hr) | (mL) | (mL/hr) | hr/kg) | AUC₀₋ₜ | AUC_{0-∞} | Cₘₐₓ |
| N | 53 | 53 | 53 | 53 | 53 | 53 | 53 | 53 | 53 | 53 | 53 | 52 | 53 |
| MEAN | 189120.87 | 202017.60 | 0.93 | 16187.96 | 6.57 | 0.10 | 7.81 | 648175.18 | 58422.07 | 716.62 | 12.05 | 12.14 | 9.58 |
| STDEV | 83556.77 | 81798.04 | 0.07 | 8018.16 | 2.99 | 0.04 | 2.77 | 335550.28 | 26228.47 | 333.57 | 0.45 | 0.42 | 0.47 |
| % CV | 44.18 | 40.49 | 7.53 | 49.53 | 45.54 | 37.65 | 35.44 | 51.77 | 44.89 | 46.55 | 3.73 | 3.42 | 4.92 |
| ME- | 185158.85 | 195366.63 | 0.94 | 14990 | 5 | 0.0893 | 7.76 | 556379.25 | 51185.81 | 632.46 | 12.12897 | 12.183299 | 9.615139 |
| DIAN | | | | | | | | | | | | | |
| MIN | 55803.45 | 59571.09 | 0.58 | 6288 | 2.5 | 0.0409 | 2.9 | 199450.34 | 22968.78 | 263.4 | 10.92959 | 10.994926 | 8.746398 |
| MAX | 430182.60 | 435373.53 | 0.99 | 42920 | 12 | 0.2391 | 16.93 | 1578014.18 | 167866.65 | 2047.15 | 12.97197 | 12.98396 | 10.66709 |

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable.

Unless defined otherwise, technical and scientific terms used herein have the same meaning as is commonly understood by one of skill in the art to which this invention belongs.

Embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An alfuzosin composition, comprising:
a tablet core comprising
alfuzosin, and
a release-retarding matrix, wherein the release-retarding matrix comprises about 40 to about 65 weight % hydroxypropyl methyl cellulose (HPMC) 2208 with a maximum apparent viscosity of about 5600 cP, based on the total weight of the tablet core; and
an extended-release coating substantially surrounding the tablet core comprising a release-retarding coating material;
wherein the alfuzosin composition administered under fed conditions is bioequivalent to a reference dosage form of New Drug Application (NDA) #021287 administered under fed conditions and
wherein the alfuzosin composition administered under fasted conditions is bioequivalent to the reference dosage form administered under fasted conditions.

2. The alfuzosin composition of claim 1, wherein the release-retarding matrix comprises about 30 to about 65 weight % HPMC 2208 with an apparent viscosity of about 3000 to about 5600 cP, based on the total weight of the tablet core.

3. The alfuzosin composition of claim 2, wherein the release-retarding matrix comprises about 35% to about 40% HPMC 2208 with an apparent viscosity of about 3000 to about 5600 cP.

4. The alfuzosin composition of claim 2, wherein the release-retarding matrix further comprises about 10 to about 20% HPMC 2208 with an apparent viscosity of about 80 to about 120 cP.

5. The alfuzosin composition of claim 1, wherein the release retarding matrix further comprises up to about 35 weight % ethyl cellulose with an ethoxyl substitution greater than about 49.5%, based on the total weight of the tablet core.

6. The alfuzosin composition of claim 1, wherein the core further comprises microcrystalline cellulose, magnesium stearate, colloidal silicon dioxide, and optionally up to about 35 weight % ethyl cellulose with an ethoxyl substitution greater than about 49.5%, based on the total weight of the tablet core.

7. The alfuzosin composition of claim 1, wherein the release-retarding coating material comprises a film forming polymer, wherein the film forming polymer is an alkylcellulose, a hydroxyalkylcellulose, a hydroxyalkyl alkylcellulose, a carboxyalkylcellulose, an alkali metal salt of a carboxyalkylcellulose, a carboxyalkyl alkylcellulose, a carboxyalkylcellulose ester, a starch, a pectin, a chitine derivate, a polysaccharide, a carrageenan, a galactomannas, traganth, agar-agar, gum arabicum, guar gum, xanthan gum, a polyacrylic acid, a polymethacrylic acid, a methacrylate copolymer, a polyvinylalcohol, polyvinylpyrrolidone, a copolymer of polyvinylpyrrolidone with vinyl acetate, a polyalkylene oxide, a copolymer of ethylene oxide and propylene oxide, or a combination comprising at least one of the foregoing film forming polymers.

8. The alfuzosin composition of claim 7, wherein the film forming polymer is methylcellulose, ethylcellulose, hydroxymethyl cellulose, hydroxyethylcellulose, hydroxypropylcellulose, hydroxybutylcellulose, hydroxyethyl methylcellulose, hydroxypropyl methylcellulose, carboxymethyl cellulose, sodium carboxymethylcellulose, carboxymethyl ethylcellulose, carboxymethylcellulose ester, sodium carboxymethylamylopectine, chitosan, alginic acid, alkali metal salt of alginic acid, ammonium salt of alginic acid, or a combination comprising at least one of the foregoing film forming polymers.

9. The alfuzosin composition of claim 1, wherein the extended-release coating is present at about 2.0 to about 20 wt. % based on the total weight of the core.

10. The alfuzosin composition of claim 9, wherein the extended-release coating is present at about 5.0 to about 16 wt. % based on the total weight of the core.

11. The alfuzosin composition of claim 1, wherein the extended-release coating comprises ethyl cellulose and hydroxypropyl methylcellulose.

12. (canceled)

13. The alfuzosin composition of claim 1, wherein the composition comprises about 10 mg alfuzosin HCl and a coating of about 6 wt. % based on the total weight of the core, and
wherein administration of a single dose of the composition to a human provides an area under the plasma alfuzosin concentration curve from time 0 to time t (AUC₀₋ₜ) of about 59 to about 444 hr*ng/ml under fed conditions or about 30 to about 239 hr*ng/ml under fasting conditions;
an area under the plasma alfuzosin concentration curve from time 0 to infinity (AUC_{0-∞}) of about 66 to about 451 hr*ng/ml under fed conditions or about 36 to about 252 hr*ng/ml under fasting conditions;
a maximum plasma alfuzosin concentration (Cₘₐₓ) of about 6.0 to about 40.0 ng/ml under fed conditions or about 2.8 to about 21.4 ng/ml under fasting conditions; or
a time to Cmax (Tmax) of about 2.5 hr to about 10.0 hr under fed conditions or about 2.0 to about 9.0 hr under fasting conditions.

14-15. (canceled)

16. The alfuzosin composition of claim 1, wherein the composition comprises about 10 mg alfuzosin HCl and a coating of about 14 wt. % based on the total weight of the core, and
wherein administration of a single dose of the composition to a human provides an area under the plasma alfuzosin concentration curve from time 0 to time t (AUC₀₋ₜ) of about 37 to about 475 hr*ng/ml under fed conditions or about 36 to about 234 hr*ng/ml under fasting conditions;
an area under the plasma alfuzosin concentration curve from time 0 to infinity (AUC_{0-∞}) of about 42 to about 492 hr*ng/ml under fed conditions or about 55 to about 250 hr*ng/ml under fasting conditions; and
a maximum plasma alfuzosin concentration (Cₘₐₓ) of about 4.5 to about 39.2 ng/ml under fed conditions or about 2.9 to about 16.5 ng/ml under fasting conditions; or
a time to Cmax (Tmax) of about 1.5 hr to about 16.0 hr under fed conditions or about 3.0 to about 24.0 hr under fasting conditions.

17-18. (canceled)

19. The alfuzosin composition of claim 1, wherein the composition exhibits under fed or fasted conditions:
a ratio of a geometric mean logarithmic transformed area under the plasma alfuzosin concentration curve from time 0 to infinity (AUC_{0-∞}) of the composition to a geometric mean logarithmic transformed AUC_{0-∞} of the alfuzosin reference dosage form of about 0.80 to about 1.25;
a ratio of a geometric mean logarithmic transformed area under the plasma alfuzosin concentration curve from time 0 to time t (AUC₀₋ₜ) of the composition to a geometric mean logarithmic transformed AUC₀₋ₜ of the alfuzosin reference dosage form of about 0.80 to about 1.25; or
a ratio of a geometric mean logarithmic transformed maximum plasma alfuzosin concentration (Cₘₐₓ) of the composition to a geometric mean logarithmic transformed Cₘₐₓ of the alfuzosin reference dosage of about 0.80 to about 1.25.

20. The alfuzosin composition of claim 19, wherein the 90% confidence limits of the ratio are 0.80 to 1.25.

21. (canceled)

22. A method of treatment, comprising
administering the composition of claim 1 to an individual in need of alfuzosin therapy.

23. The method of claim 22, wherein the individual has benign prostatic hyperplasia.

24. (canceled)

25. An alfuzosin composition comprising
a tablet core comprising
alfuzosin and a release-retarding matrix comprising
about 35 to about 40% (by weight) hydroxypropyl methyl cellulose 2208 having a viscosity of about 3000 to about 5600 cP;
about 10 to about 40% (by weight) hydroxypropyl methyl cellulose 2208 having a viscosity of about 80 to about 120 cP; and
up to 35% (by weight) ethyl cellulose with an ethoxyl substitution greater than about 49.5%, wherein the weight percents are based on the total weight of the tablet core; and
an extended-release coating substantially surrounding the tablet core comprising a release-retarding coating material;
wherein the alfuzosin composition is bioequivalent to a reference dosage form of New Drug Application (NDA) #021287.

26-27. (canceled)

28. An alfuzosin composition, comprising:
a tablet core comprising
alfuzosin, and
a release-retarding matrix, wherein the release-retarding matrix comprises about 40 to about 65 weight % hydroxypropyl methyl cellulose (HPMC) 2208 with a maximum apparent viscosity of about 5600 cP, based on the total weight of the tablet core; and
an extended-release coating substantially surrounding the tablet core comprising a release-retarding coating material;
wherein about 10% to about 17% of the alfuzosin is dissolved in 1 hour;
about 25% to about 40% of the alfuzosin is dissolved in 3 hours;
about 50% to about 70% of the alfuzosin is dissolved in 8 hours; and
no less than about 80% of the alfuzosin is dissolved in 23 hours,
wherein dissolution is determined according to USP 28 <711> test apparatus 2 (paddle) with a 100 rpm paddle speed at 37 C using Sandwich sinkers in sequential dissolution media as follows: 0.01 M HCl at 0-1 hr, pH 4.5 acetate buffer at 2-3 hr, and pH 6.8 phosphate buffer at 9-23 hr.

29. The alfuzosin composition of claim 28, wherein the alfuzosin composition has a dissolution profile that is substantially identical to a dissolution profile of an equivalent strength of a reference dosage form of New Drug Application (NDA) #021287.

30-34. (canceled)
